(19) United States
(12) Patent Application Publication
(10) Pub. No.: US 2020/0345846 A1
(43) Pub. Date: Nov. 5, 2020

(54) CORROLE COMPOSITIONS

(71) Applicant: TECHNION RESEARCH & DEVELOPMENT FOUNDATION LIMITED, Haifa (IL)

(72) Inventors: Zeev GROSS, Petach Tikva (IL); Matan SOLL, Kibbutz Rammat Yohannan (IL)

(73) Assignee: TECHNION RESEARCH & DEVELOPMENT FOUNDATION LIMITED, Haifa (IL)

(21) Appl. No.: 16/643,569

(22) PCT Filed: Aug. 30, 2018
(86) PCT No.: PCT/IL2018/050967

§ 371 Date: Mar. 1, 2020

Related U.S. Application Data
(60) Provisional application No. 62/552,602, filed on Aug. 31, 2017.

(51) Int. Cl. A61K 41/00; A61K 47/64; A61K 51/04; A61K 51/08; A61K 47/69; A61P 35/00

(52) U.S. Cl. CPC A61K 41/0057; A61K 47/643; A61K 51/0485; A61P 35/00; A61K 51/081; A61K 51/088; A61K 47/6929; A61K 51/0497

(57) ABSTRACT

Embodiments of the invention relate to compositions comprising a corrole and optionally a protein, wherein the composition are characterized by improved water solubility. The corrole may be a hydrophobic corrole, according to formula [I]. The protein may be a plasma protein. The plasma protein may be albumin. The compositions may be in nanoparticulate form.

### CROSS REFERENCE TO RELATED APPLICATIONS

This application claims the benefit of priority to U.S. Provisional Patent Application No. 62/552,602 filed Aug. 31, 2017, the contents of which are incorporated herein by reference in their entirety.

### TECHNICAL FIELD

Embodiments of the invention relate to compositions comprising corroles.

### BACKGROUND

Corroles are organic molecules having a contracted porphyrin ring comprising nineteen carbon atoms and 4 nitrogen atoms, and are capable of binding transition metals. Various transition metal complexes of corroles were found to have physiological effects in mammals including but not limited to antioxidant or anticancer effects.

### SUMMARY

Embodiments of the invention relate to compositions comprising at least one corrole and at least one protein or peptide. In some embodiments, a composition in accordance to an embodiment of the invention comprises a conjugate of a protein with a corrole. In some embodiments, the conjugate is arranged so that the corrole is located internally to a layer of plasma protein surrounding the corrole. In some embodiments, a conjugate is in nanoparticulate form.

In some embodiments, the protein is a plasma protein. The plasma protein may be selected from the group consisting of albumin, lipoprotein, glycoprotein, and α, β, and γ globulin. In some embodiments, the albumin is selected from the group consisting of human serum albumin or bovine serum albumin, and egg albumin. In some embodiments, the protein is transferrin. In some embodiments, the protein is a recombinant protein.

Compositions according to embodiments of the invention are advantageous when compared to the corrole at least because of improved water solubility.

According to an embodiment of the invention, the corrole is a hydrophobic corrole. In some embodiments, a corrole has a structure according to formula [I]:

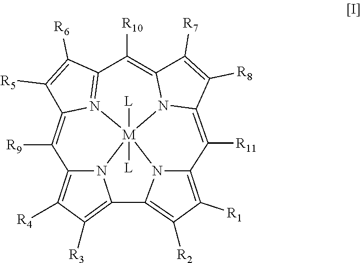

wherein R₁ through R₈ are each independently H, halogen, C₁-C₄ alkyl or halogenated alkyl; R₉ through R₁₁ are each independently H, straight or branched C₁-C₁₂ alkyl, C₆-C₁₂ aralkyl, C₆-C₁₂ aryl, or C₆-C₁₂ heteroaryl, having an oxygen or nitrogen atom as a heteroatom, wherein each of straight or branched C₁-C₁₂ alkyl, C₆-C₁₂ aralkyl, C₆-C₁₂ aryl, or C₆-C₁₂ heteroaryl may be unsubstituted or substituted by a halogen or by an amino group; L, may be present or absent, if present is halogen, an oxo group, a pyridine group or a water molecule;
and
M is H or an atom from group 3-15 or an f-block element.

In some embodiments, M is a contrast agent.

In some embodiments, R₁-R₈ are each independently CO₂H, CO₂R, CHO, hydrogen or halogen

In some embodiments, a corrole has a solubility in water of less than 0.1 mg/L.

In some embodiments, a protein is non-covalently conjugated to a corrole. In some embodiments, a composition according to some embodiments of the present invention is in the form of nanoparticles. In some embodiments, at least 50% of particles have a mean diameter size in the range of 30 nm to 200 nm.

The inventors have found that nanoparticles may be formed that comprise the aforementioned corroles and proteins. The nanoparticles preferably have a mean particle diameter of between about 10 nanometers (nm) and 200 (nm) preferably between 50 and 150 nm, when measured using electron microscopy.

The nanoparticles may comprise protein and a corrole in a molar ratio (protein:corrole) of between 100:1 and 0.1:1. Optionally, the molar ratio (protein:corrole) may be between 10:1 and 1:1.

In some embodiments, a composition according to the present invention has solubility in water the range of 0.5 mg/L to 50 g/L.

Additional embodiments of the invention relate to methods of manufacture of compositions and/or nanoparticles comprising a protein and a corrole.

Additional embodiment of the invention relate to uses and methods of treatment using the compositions comprising a protein and a corrole.

According to an aspect of the present invention, there is provided a method for treating a disease in a subject in need thereof. In some embodiments, there is provided a method for treating a disease in a subject in need thereof comprising administering a therapeutically effective amount of a composition according to the present invention to a subject, thereby treating a disease. In some embodiments, the disease is cancer. In some embodiments, the disease is associated with a reactive oxygen species.

According to an aspect of the present invention, there is provided a method for imaging a condition in a subject. In some embodiments, there is provided a method for imaging a condition in a subject comprising administering an effective amount of a composition according to the present invention to the subject, wherein M is an imaging agent and applying a magnetic field, X-radiation, UV-vis light or any combination thereof to the subject, thereby imaging the condition in the subject. In some embodiments, imaging is MRI imaging, fluorescence imaging, positron-emission tomography (PET), or any combination thereof.

According to an aspect of the present invention, there is provided a method for imaging and diagnosing a disease in a subject, further comprising quantifying a signal intensity of an image of the subject and comparing the signal intensity with a reference. In some embodiments, signal intensity is greater than the reference is indicative of the subject being afflicted with a disease.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF FIGURES

Non-limiting examples of embodiments are described below with reference to figures attached hereto that are listed following this paragraph. Identical structures, elements or parts that appear in more than one figure are generally labeled with a same numeral in all the figures in which they appear, and a numeral labeling an icon representing a given feature in a figure may be used to reference the given feature. Dimensions of components and features shown in the figures are chosen for convenience and clarity of presentation and are not necessarily shown to scale.

FIGS. 1A-1G show chromatograms depicting circular dichroism of corrole nanoparticles prepared according to embodiments of the invention comprising corroles and a protein (bovine serum albumin, BSA);

FIG. 1H shows a chromatogram depicting circular dichroism of a sample comprising BSA alone, without a corrole;

FIG. 1I shows the induced CD spectrum recorded for (2)Mn/BSA NPs and FIG. 1J shows asymmetric oxidation of thioanisole by H₂O₂ in solutions that contained NPs composed of HSA with Fe(III)/Mn(III)corroles;

FIG. 1K shows Cryo-TEM images of isolated (2)Au/BSA (“b” and “c” denote two different magnifications) and of similarly treated BSA but without corrole (denoted as “d”);

FIG. 1L presents molecular structures of the investigated corroles;

FIG. 1M shows the selectivity of (1)M (red bars) and (2)M (black bars) complexes to the VLDL fraction of human serum.

FIG. 2A depicts cytotoxic effect of various concentrations of corrole nanoparticles comprising corrole 1-Au and BSA on pancreatic cancer cells, compared to a control comprising only BSA without a corrole;

FIG. 2B depicts cytotoxic effect of various concentrations of corrole nanoparticles comprising corrole 2-Au and BSA on pancreatic cancer cells, compared to a control comprising only BSA without a corrole;

FIG. 3 is a graph showing fluorescence detection limit in sera of Corrole 5-Ga;

FIGS. 4A-B are graphs showing PK profiles of Corrole 4-Ga, Corrole 1-Ga and Corrole 5-Ga (1M, 3M and 5M respectively) at the 10 mg/kg dose both on a normal (FIG. 4A) and logarithmic scale (FIG. 4B);

FIGS. 5A-C are representative cryo-TEM images of Corrole 5-Ga/Human serum albumin (HSA) nanoparticles (NP's) with high magnification (FIG. 5A) and lower magnification (FIG. 5B) and distribution analysis of particle size analyzed from 12 separate fields in two distinct samples of Corrole 5-Ga/HSA NP's (FIG. 5C);

FIGS. 6A-C are representative cryo-TEM images of Samples of sera from mouse treated with Corrole 5-Ga/Human serum albumin (HSA) nanoparticles (NP's) (FIG. 6A) and vehicle at time points 30 minutes (FIG. 6B) and a graph showing the distribution analysis of particle size analyzed from 12 separate fields in two distinct samples of Corrole 5-Ga/HSA NP's treated mice (FIG. 6C);

FIGS. 7A-E are live time laps imaging of DU-145 cell line (series ranging from time points t=0, 3, 6 and 10 minutes) incubated with the calcium indicator Fluo-8 AM 4 μM for 30 min prior to washings and the addition of: 2 μM of Corrole 5-Ga/HSA NP's (FIG. 7A), 20 μM of Corrole 5-Ga/HSA NP's (FIG. 7B), HSA control (FIG. 7C) and graphs showing the quantification of fluorescence from each cell in the 12 separated fields obtained from: series 7A, 2 μM of Corrole 5-Ga/HSA NP's (FIG. 7D) and series 7B, 20 μM of Corrole 5-Ga/HSA NP's (FIG. 7E); data points are presented as mean±SEM n=620. Fluorescence was recorded using a ×40 objective and an IN CELL GE analyzer supported with in cell 2000 software. Samples were excited using a FITC filter for Fluo-8 AM detection. Representative images of 12 separate fields, representative results of 3 distinct repeats;

FIGS. 8A-D are live time laps imaging of DU-145 cell line (series ranging from time points t=0, 3, 6 and 10 minutes) incubated with the calcium indicator Fluo-8 AM 4 μM for 30 min prior to washings and the addition of: 2 μM of Corrole 5-Ga/HSA NP's followed by excitation of Corrole 5-Ga at each measured time point (FIG. 8A), 2 μM of Corrole 5/HSA NP's (FIG. 8B), 20 μM of Corrole 5/HSA NP's (FIG. 8C), and quantification of calcium indicator Fluo-8 AM fluorescence obtained from each cell in the 12 separated fields of 8A (FIG. 8D); data points are presented as mean±SEM n=550. Fluorescence was recorded using a ×40 objective and an IN CELL GE analyzer supported with in cell 2000 software. Samples were excited using a FITC filter for Fuo-8 AM detection and a CFP filter for Corrole 5-Ga detection and excitation.

FIGS. 9A-B are live time laps imaging of Corrole 5-Ga/HSA NP's uptake in to DU-145 (prostate cancer) cell line: series of images taken at time intervals of 30 sec raging from t=0 upper left corner to t=6:30 min lower right corner, time point is indicated in white on upper left corner of each image (FIG. 9A); quantification of fluorescence from the center of each cell in the 12 separated fields obtained, data points are presented as mean±SEM n=560 (FIG. 9B). Fluorescence was recorded using a ×40 objective and an IN CELL GE analyzer supported with in cell 2000 software. Samples were excited using a CFP filter for Corrole 5-Ga detection. Representative images of 12 separate fields, representative results of 3 distinct repeats.

FIG. 10 shows live time laps imaging of Corrole 5/HSA BNC's uptake in to DU-145 cell line: series of images (originally taken at time intervals of 30 sec) raging from t=0 upper left corner to t=10 min lower right corner, time point is indicated in white on upper left corner of each image; representative images of chosen time points. Fluorescence was recorded using a ×40 objective and an IN CELL GE analyzer supported with in cell 2000 software. Samples were excited using a CFP filter for corrole 5 detection. Representative images of 12 separate fields, representative results of 3 distinct repeats;

FIGS. 11A-B are HPLC chromatograms with reading set at 280 nm for detection of BSA and at 416 nm for detection of the corrole 5-P(OH)2 (FIG. 11A); and UV-vis spectra recorded for the HPLC fractions obtained for the corrole 5-P(OH)2/HSA conjugate (FIG. 11B);

FIGS. 12A-C are crystal structures of corrole 5-Ga (pyridine) (FIG. 12A), corrole 5-Mn (DMF) (FIG. 12B), and corrole 5-P (F2) (FIG. 12C); and

FIG. 13 is a graph showing the In Vivo efficacy against a triple negative cell line MDA-MB-231 implanted in nude mice.

FIG. 14A presents images showing uptake of 5-Ga albumin base nanoparticles by DU-145 prostate cell line dyed with 2 μM mitotracker green (MTG) imaged after 2 h incubation (black arrows designated apoptotic cells with obvious apoptotic blabbing phenotype): control (denoted as “a”); 5-Ga 20 μM, depolarization of mitochondria is apparent (indicative of apoptosis) due to no fluorescence from MTG, lysosomal fusion with the contained compound is also apparent (also indicative of apoptosis) (denoted as “b”); 5-Ga 5 μM, abnormal mitochondrial signal is apparent with some co-localization of 5-Ga with MTG (yellow signal, denoted as “c”).

FIG. 14B presents graphs showing IC50 values of 5-Ga added dissolved in DMSO only assessed by MTT test (denoted as “a”) and annexin-FITC apoptotic assay kit (absolute proof of apoptosis) after 24 h incubation (denoted as “b”).

FIGS. 15A-G present graphs showing fluorescence-activated cell sorting (FACS) analysis of DU-145 prostate cancer cell line treated with an annexin V-FITC kit after 4 h incubation with: HSA control (FIG. 15A); 5-Ga HSA NP's 2, 5, 10, 15, 20 μM, respectively (FIGS. 15B-F). Plotting of gated cells (within Ml) positive to FITC (i.e., apoptosis) against 5-Ga concentration and assessment of IC50 value (IC50=6.85 μM) (FIG. 15G).

FIGS. 16A-G present graphs showing FACS analysis of DU-145 prostate cancer cell line treated with a mitochondrial depolarization reporter (another indication for apoptosis) kit after 4 h incubation with: HSA control (FIG. 16A); CCCP (positive control which induces mitochondrial depolarization) (FIG. 16B); and 5-Ga NP's 2, 5, 10, 15, 20 μM, respectively (FIGS. 16C-G). Arrow pointing towards the right quarter indicates percentage of depolarization.

FIGS. 17A-G present graphs showing FACS analysis of DU-145 prostate cancer cell line treated with an annexin V-FITC kit after 24 h incubation with: BSA control (FIG. 17A); 0-lapachone (a known inducer of apoptosis, note the strong shift of the distribution curve towards stronger mean fluorescence) (FIG. 17B; and 1-Ga HSA NP's 0.001, 0.01, 0.1, 1, 10 μM, respectively (FIGS. 17C-G). Analysis of P-lapachone was done after 4 h since it induces apoptosis faster. In contrast with 5-Ga, even when the protein formulation is used there is no induction of apoptosis with 1-Ga (The same is when it is added using DMSO, Table 3).

FIGS. 18A-G present graphs showing FACS analysis of DU-145 prostate cancer cell line treated with a mitochondrial depolarization reporter kit (similar to FIGS. 16A-G) after 24 h incubation with: BSA control (FIG. 18A); CCCP (positive control) (FIG. 18B); and 1-Ga HSA NP's 0.001, 0.01, 0.1, 1, 10 μM, respectively (FIGS. 18C-G). Arrow pointing towards the right quarter indicates percentage of depolarization. In contrast with 5-Ga, no apparent mitochondrial depolarization could be detected even under the use of the aforementioned formulation.

FIG. 19 presents comparative graphs showing the effect of encapsulation on the emission of virtually identical concentrations of (2)Ga in DMSO (blue trace; “1”), dialyzed NPs formed with BSA (red trace; “2”), treatment of the latter solution with 2% Triton-X (green trace; “3”), and extraction of (2)Ga from the last solution into dichloromethane (black trace; “4”). The instrumental parameters were the same in all cases.

### DETAILED DESCRIPTION

According to one aspect, the present invention provides a composition comprising a corrole according to formula [I];

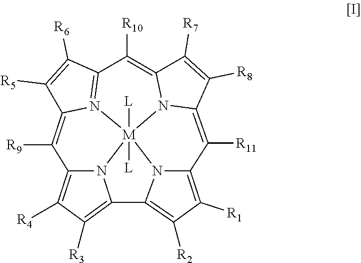

wherein, R1 through R8 are each independently H, halogen, C1-C4 alkyl or halogenated alkyl; R9 through R11 are each independently H, straight or branched C1-C12 alkyl, C6-C12 aralkyl, C6-C12 aryl, or C6-C12 heteroaryl, having an oxygen or nitrogen atom as a heteroatom. In some embodiments, each of straight or branched C1-C12 alkyl, C6-C12 aralkyl, C6-C12 aryl, or C6-C12 heteroaryl is unsubstituted. In some embodiments, each of straight or branched C1-C12 alkyl, C6-C12 aralkyl, C6-C12 aryl, or C6-C12 heteroaryl is substituted by a halogen or by an amino group.

In some embodiments, L is present. In some embodiments, L is selected from a halogen, an oxo group, a hydroxyl group, acetonitrile, dimethylsulfoxide, dimethylformamide a pyridine group or a water molecule. In some embodiments, L is selected from amino acids such as glutamate or histidine, biotin, carbohydrates such as glucose, oligosaccharides and lipids such as sphingosine, ganglioside. In some embodiments, L is absent.

In some embodiments, M is H. In some embodiments, M is an atom from group 3-15. In some embodiments, M is an f-block element.

According to some embodiments, the present invention provides a composition comprising a corrole according to formula [I], and a protein non-covalently conjugated to the corrole. In some embodiments, the composition has the form of nanoparticles.

In some embodiments, at least 50% of particles according to the present invention have a mean diameter size in the range of 30 nm to 200 nm. In some embodiments, at least 60%, at least 65%, at least 70%, at least 75%, at least 80%, at least 85%, or at least 90% of the particles have a mean diameter size in the range of 30 nm to 200 nm, including any value therebetween.

In some embodiments, the corrole is hydrophobic.

As used herein the term “hydrophobic” refers to compounds or compositions which have very low water solubility. In some embodiments, a hydrophobic compound or composition has a solubility in water lower than 1 mg/L.

In some embodiments, the corrole has a solubility in water of less than 10 mg/L. In some embodiments, the corrole has a solubility in water of less than 9 mg/L, less than 8 mg/L, less than 7 mg/L, less than 6 mg/L, less than 5 mg/L, less than 4 mg/L, less than 3 mg/L, less than 2 mg/L, less than 1 mg/L, less than 0.7 mg/L, less than 0.5 mg/L, or less than 0.1 mg/L, including any value and range therebetween.

In some embodiments, non-covalent forces refer to van der Waals, steric, hydrogen bonding, hydrophobic, electrostatic and metal-ligand interactions.

According to some embodiments, the present invention provides a composition in the form of nanoparticles. In some embodiments, a nanoparticle comprises a corrole according to formula [I], and a protein.

In some embodiments, nanoparticles have a mean particle diameter in the range of about 10 nanometers (nm) to 200 (nm). In some embodiments, nanoparticles have a mean particle diameter in the range of 50 nm to 150 nm, 50 nm to 200 nm, 10 nm to 150 nm, 70 nm to 150 nm, 80 nm to 150 nm, 50 nm to 190 nm, 50 nm to 180 nm, 50 nm to 170 nm, 40 nm to 150 nm, 40 nm to 100 nm, 30 nm to 150 nm, 30 nm to 100 nm or 50 nm to 160 nm, including any range therebetween.

In some embodiments, a nanoparticle as described herein comprises a core and a shell. In some embodiments, a corrole is a core. In some embodiments, a protein is a shell. In some embodiments, a shell is surrounding a core. In some embodiments, a protein is surrounding a corrole. In some embodiments a corrole is located internally to a layer of a protein surrounding the corrole.

In some embodiments, a core has a size in the range of 20 nm to 60 nm. In some embodiments, a core has a size in the range of 25 nm to 60 nm, 30 nm to 60 nm, 35 nm to 60 nm, 40 nm to 60 nm, 20 nm to 50 nm, 20 nm to 40 nm, 25 nm to 40 nm or 30 nm to 40 nm, including any range therebetween.

In some embodiments, a shell has a thickness of about 10 nm to 70 nm. In some embodiments, a protein-based shell has a thickness of about 10 nm to 60 nm, 10 nm to 50 nm, 10 nm to 40 nm, about 15 nm to 70 nm, or about 20 nm to 70 nm, including any range therebetween.

In some embodiments, a nanoparticle shell is at least partially surrounding the nanoparticle core. In some embodiments, a shell is surrounding the core at least 80% of the total surface of the core. In some embodiments, a protein-based shell is partially surrounding a bioactive compound at least 85%, 87%, at least 90%, at least 93%, at least 95%, at least 98%, or at least 98% of the total surface of the core, including any value therebetween.

In some embodiments, a nanoparticle sell is at least partially surrounding the nanoparticle core about 85% to 100% of the total surface of the core. In some embodiments, a shell is partially surrounding a core about 85% to 99%, 85% to 98%, 85% to 95%, 85% to 90%, 85% to 89%, or 85% to 70% of the total surface of the core, including any range therebetween.

In some embodiments, R1-R8 are each independently H. In some embodiments, R1-R8 are each independently halogen. In some embodiments, R1-R8 are each independently C1-C4 alkyl. In some embodiments, R1-R8 are each independently halogenated alkyl.

In some embodiments, R9-R11 are each independently C6 aryl. In some embodiments, R9-R11 are each independently C6 aryl substituted by one or more halogen groups. In some embodiments, R9-R11 are each independently C6 aryl substituted by one or more halogen groups and amino groups. In some embodiments, R9-R11 are each independently C6 aryl substituted by amino groups.

In some embodiments, R9-R11 are each C6F5 and R1-R8 are each H. In some embodiments, R9-R11 are each C6F4NH2 and R1-R8 are each H. In some embodiments, R9-R11 are each C6F5 and R1-R4 are each I and R5-R8 are each H. In some embodiments, R9-R11 are each CF3 and R1-R8 are each H.

In some embodiments, M is a transition metal. In some embodiments, M is an f-block element. In some embodiments, M is selected from the group consisting of: Sb, Sn, Al, Ga, Mn, Gd, Fe, Au, P and Al.

As used herein, the term “metal” refers to those elements that are in the d-block and f-block of the Periodic Chart of the Elements, along with those elements that have metal-like properties, such as silicon and germanium.

As used herein, the term “d-block” refers to those elements that have electrons occupying the 3d, 4d, 5d, and 6d orbitals surrounding the nucleus of the element.

As used herein, the term “f-block” refers to those elements that have electrons occupying the 4f and 5f orbitals surrounding the nucleus of the element, including the lanthanides and the actinides.

In some embodiments, the molar ratio between the protein and the corrole is in the range of 100:1 to 0.1:1. In some embodiments, the molar ratio between the protein and the corrole is in the range of 90:1 to 0.1:1, 80:1 to 0.1:1, 70:1 to 0.1:1, 60:1 to 0.1:1, 50:1 to 0.1:1, 40:1 to 0.1:1, 30:1 to 0.1:1, 20:1 to 0.1:1, or 10:1 and 0.1:1, including any range therebetween.

In some embodiments, a protein is a plasma protein. In some embodiments, a plasma protein is selected from the group consisting of: albumin, lipoprotein, glycoprotein, and α, β, and γ globulin. In some embodiments, a plasma protein comprises albumin. In some embodiments, a plasma protein comprises albumin selected from the group consisting of: human serum albumin and bovine serum albumin.

As used herein, the term “plasma” refers to the fluid, non-cellular portion of the blood of humans or animals as found prior to coagulation.

As used herein, the term “plasma protein” refers to the soluble proteins found in the plasma of normal humans or animals. These include but are not limited to coagulation proteins, albumin, lipoproteins and complement proteins.

As used herein, the term “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

According to some embodiments of the present invention, a composition as described herein has a solubility in water of at least 0.5 mg/L. In some embodiments, a composition as described herein has a solubility in water of at least 0.5 mg/L, 1 mg/L, 5 mg/L, 20 mg/L, 50 mg/L, 60 mg/L, 63 mg/L, or 100 mg/L, including any value therebetween.

In some embodiments, a composition has a solubility in water in the range of 0.5 mg/L to 50 g/L. In some embodiments, a composition has a solubility in water in the range of 0.5 mg/L to 45 g/L, 0.5 mg/L to 40 g/L, 0.5 mg/L to 35 g/L, 0.5 mg/L to 30 g/L, 1 mg/L to 50 g/L, 5 mg/L to 50 g/L, 10 mg/L to 50 g/L, 20 mg/L to 50 g/L, 50 mg/L to 50 g/L, 60 mg/L to 50 g/L, 63 mg/L to 50 g/L, or 100 mg/L to 50 g/L, including any range therebetween.

In some embodiments, the present invention is directed to a composition for use in the manufacture of a medicament. In some embodiments, the medicament is for treating a disease. In some embodiments, the disease is cancer. In some embodiments, the disease is associated with a reactive oxygen species.

In some embodiments, the present invention is directed to a composition for use in the manufacture of pharmaceutical compositions. In some embodiments, the composition is a pharmaceutical composition. In some embodiments, the composition is a pharmaceutical composition comprising a pharmaceutically acceptable excipient and a therapeutically effective amount of a corrole nanoparticle as described elsewhere herein.

As used herein the term “pharmaceutically acceptable excipient” refers to an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In some embodiments, a pharmaceutical composition contains a pharmaceutically acceptable carrier. As used herein the term “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some embodiments, a pharmaceutical composition is formulated for delivery via any route of administration. As used herein the term “route of administration” refers to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

In some embodiments, a composition as described herein is a contrast agent. In some embodiments, a composition as described herein is a contrast agent selected from the group consisting of T1-class and T-2 class MRI contrast agent.

In some embodiments, the present invention is directed to a composition for use in magnetic resonance imaging (MRI).

The phrase “MRI contrast agents” refers to a group of contrast media typically used to improve the visibility of internal body structures in magnetic resonance imaging.

The phrase “T1-class and T2-class MRI contrast agents” is used herein to denote that tissue can be characterized by two different relaxation times, typically referred to as T1 and T2. T1 (longitudinal relaxation time) is known to a skilled artisan as the time constant which determines the rate at which excited protons return to equilibrium. It is a measure of the time taken for spinning protons to realign with the external magnetic field. T2 (transverse relaxation time) is known to a skilled artisan as the time constant which determines the rate at which excited protons reach equilibrium or go out of phase with each other. It is a measure of the time taken for spinning protons to lose phase coherence among the nuclei spinning perpendicular to the main field.

As used herein, the term “radiocontrast agent” refers to a group of contrast media typically used to improve the visibility of internal body structures in X-ray based imaging techniques.

As used herein, the term “X-ray based imaging techniques” refers to a group of medical imaging technics that make use of X-radiation such as computed tomography (CT) where tomographic images or slices of specific areas of the body are obtained from a large series of two-dimensional X-ray images taken in different directions.

As used herein the term “positron-emission tomography” (PET) refers to a nuclear medicine functional imaging technique that is used to observe metabolic processes in the body as an aid to the diagnosis of disease. The system detects pairs of gamma rays emitted indirectly by a positron-emitting radionuclide, most commonly 18F which is introduced into the body on a biologically active molecule called a radioactive tracer. Three-dimensional images of tracer concentration within the body are then constructed by computer analysis. In modern PET-CT scanners, three-dimensional imaging is often accomplished with the aid of a CT X-ray scan performed on the patient during the same session, in the same machine.

In some embodiments, the present invention is directed to a composition for use in photodynamic therapy. In some embodiments, the present invention is directed to a composition for use in asymmetric catalysis. In some embodiments, the present invention is directed to a composition for use in Dye Sensitized Solar Cells. In some embodiments, the present invention is directed to a composition for use in optical imaging of cells. In some embodiments, the present invention is directed to a composition for use in sonodynamic therapy.

The method:

According to an aspect of some embodiments of the present invention there is provided a method for treating a disease in a subject in need thereof. In some embodiments the method comprises administering a therapeutically effective amount of a composition according to the present invention to a subject, thereby treating a disease.

In some embodiments, there is provided a method for treating a disease in a subject in need thereof, comprising administering a therapeutically effective amount of a composition comprising a corrole according to formula [I], wherein R1 through R8 are each independently H, halogen, C1-C4 alkyl or halogenated alkyl, R9 through R11 are each independently H, straight or branched C1-C12 alkyl, C6-C12 aralkyl, C6-C12 aryl, or C6-C12 heteroaryl, having an oxygen or nitrogen atom as a heteroatom, wherein each of straight or branched C1-C12 alkyl, C6-C12 aralkyl, C6-C12 aryl, or C6-C12 heteroaryl may be unsubstituted or substituted by a halogen or by an amino group, L, may be present or absent, if present is halogen, an oxo group, a hydroxyl group, a pyridine group or a water molecule, and M is H or an atom from group 3-15 or an f-block element, to a subject, thereby treating a disease. In some embodiments, the corrole is non-covalently conjugated a protein. In some embodiments, the composition having the form of nanoparticles.

Method of Treating Cancer:

In some embodiments, there is provided a method for treating cancer in a subject in need thereof, comprising administering a therapeutically effective amount of a composition according to the present invention to a subject, thereby treating cancer.

According to some embodiments, the present invention provides a composition comprising a corrole according to formula [I], wherein, R1 through R8 are each independently H, halogen, C1-C4 alkyl or halogenated alkyl; R9 through R11 are each independently a straight or branched C1-C12 substituted by a halogen. In some embodiments, L is absent. In some embodiments, M is selected from Ga or P.

In some embodiments, there is provided a method for treating cancer in a subject in need thereof, comprising administering a therapeutically effective amount of a composition comprising a corrole according to formula [I], wherein, R1 through R8 are each independently H, halogen, C1-C4 alkyl or halogenated alkyl; R9 through R11 are each independently a straight or branched C1-C12 substituted by a halogen, L is absent, M is selected from Ga or P, to a subject, thereby treating cancer.

In some embodiments, there is provided a method for treating a disease associated with a reactive oxygen species.

In some embodiments, there is provided a method for photodynamic therapy (PDT). In some embodiments, there is provided a method for sonodynamic therapy (SDT). In some embodiments, there is provided a method for photodynamic therapy (PDT) and sonodynamic therapy (SDT). In some embodiments, there is provided a method for photodynamic therapy (PDT) comprising the steps of administering a therapeutically effective amount of a composition according to the present invention to a subject, and irradiating the subject with energy source at a wavelength capable of exciting the composition. In some embodiments, a wavelength capable of exciting the composition is capable of achieving tissue-penetration.

In some embodiments, there is provided a method for sonodynamic therapy (SDT), comprising the steps of administering a therapeutically effective amount of a composition according to the present invention to a subject, and irradiating the subject with energy source at a wavelength capable of exciting the composition.

In some embodiments, there is provided a method for photodynamic therapy (PDT) and sonodynamic therapy (SDT), comprising the steps of administering a therapeutically effective amount of a composition according to the present invention to a subject, and irradiating the subject with energy source at a wavelength capable of exciting the composition.

In some embodiments, the energy source is selected from light, ultrasound, magnetic force, electromagnetic radiation, LEDs or lasers in the UV-vis electromagnetic spectrum or near infrared.

In some embodiments, the wavelength is between 350 nm to about 900 nm. In some embodiments, the wavelength is between 400 nm to about 900 nm, 450 nm to about 900 nm, 500 nm to about 900 nm, 550 nm to about 900 nm, 600 nm to about 900 nm, or 650 nm to about 900 nm, including any range therebetween.

In some embodiments, irradiating is a plurality of times. In some embodiments, a plurality of times is to achieve treatment effect.

As used herein, the term “sonodynamic therapy” refers to a method involving the combination of ultrasound and a sonosensitiser in which activation of the sonosensitiser by acoustic energy results in the generation of reactive oxygen species, such as singlet oxygen.

As used herein, the term “photodynamic therapy” refers to a process whereby light of a specific wavelength is directed to tissues undergoing treatment or investigation that have been rendered photosensitive through the administration of a photoreactive or photosensitizing agent. The objective may be either diagnostic, where the wavelength of light is selected to cause the photoreactive agent to fluoresce, thus yielding information about the tissue without damaging the tissue, or therapeutic, where the wavelength of light delivered to the target tissue under treatment causes the photoreactive agent to undergo a photochemical interaction with oxygen in the tissue under treatment that yields high energy species, such as singlet oxygen, causing local tissue lysing or destruction.

As used herein, the term “photosensitizer” or “photosensitizing agent” refers to a chemical compound that upon exposure to photoactivating light is activated, converting the photosensitizing agent itself, or some other species, into a cytotoxic form, whereby target cells are killed or their proliferative potential diminished. Thus, photosensitizing agents may exert their effects by a variety of mechanisms, directly or indirectly. For example, certain photosensitizing agents become directly toxic when activated by light, whereas others act to generate toxic species, e.g. oxidizing agents such as singlet oxygen or oxygen-derived free radicals, which are extremely destructive to cellular material and biomolecules such as lipids, proteins and nucleic acids. Psoralens are exemplary of directly acting photosensitizers; upon exposure to light they form adducts and cross-links between the two strands of DNA molecules, thereby inhibiting DNA synthesis. Porphyrins are exemplary of photosensitizing agents that act indirectly by generation of toxic oxygen species. Virtually any chemical compound that, upon exposure to photoactivating light, is converted into or gives rise to a cytotoxic form may be used in this invention. Generally, the chemical compound is nontoxic to the animal to which it is administered or is capable of being formulated in a nontoxic composition, and the chemical compound in its photodegraded form is also nontoxic.

As used herein, the term “irradiating” and “irradiation” refers to exposing a subject to all wavelengths of light. In some embodiments, the irradiating wavelength is selected to match the wavelength(s) which excite the photosensitive compound. In some embodiments, the radiation wavelength matches the excitation wavelength of the photosensitive compound and has low absorption by the non-target tissues of the subject, including blood proteins.

Irradiation is further defined herein by its coherence (laser) or non-coherence (non-laser), as well as intensity, duration, and timing with respect to dosing using the photosensitizing compound. The intensity or fluence rate must be sufficient for the light to reach the target tissue. The duration or total fluence dose must be sufficient to photoactivate enough photosensitizing compound to act on the target tissue.

According to an aspect of some embodiments of the present invention there is provided a method for imaging a condition in a subject. In some embodiments, the method comprises administering an effective amount of a composition according to the present invention to the subject, wherein M is an imaging agent and applying a magnetic field, X-radiation, UV-vis light or any combination thereof to the subject, thereby imaging the condition in the subject.

In some embodiments, imaging is MRI imaging, fluorescence imaging, positron-emission tomography (PET), or any combination thereof.

According to an aspect of some embodiments of the present invention there is provided a method for imaging a condition in a subject and diagnosing a disease in a subject. In some embodiments, the method comprises, administering an effective amount of a composition according to the present invention to the subject, wherein M is an imaging agent and applying a magnetic field, X-radiation, UV-vis light or any combination thereof to the subject, quantifying a signal intensity of an image of the subject, and comparing the signal intensity with a reference, wherein said signal intensity is greater than the reference is indicative of the subject being afflicted with a disease, thereby diagnosing a disease in the subject.

In some embodiments, a reference is a predetermined signal intensity indicative of a healthy subject.

In some embodiments, compositions described herein are radiophores and emit radiation that is useful in diagnostic and/or monitoring methods employing positron emission tomography. The compounds emit positron radiation capable of producing a pair of annihilation photons moving in opposite directions, the annihilation photons are produced as a result of positron annihilation with an electron. In some embodiments, the radiophore is a radioisotope linked to another chemical structure.

In some embodiments, the radiophore includes a positron-emitting isotope having a suitable half-life and toxicity profile. In some embodiments, the radioisotope has a half-life of more than 30 minutes, more than 70 minutes, more than 80 minutes, more than 90 minutes, more than 100 minutes. In some embodiments, the radioisotope has a half-life of about 30 minutes to about 4 hours, about 70 minutes to about 4 hours, about 80 minutes to about 4 hours, about 90 minutes to about 4 hours, about 100 minutes to about 4 hours, about 30 minutes to about 6 hours, about 70 minutes to about 6 hours, about 80 minutes to about 6 hours, about 90 minutes to about 6 hours, about 100 minutes to about 6 hours, about 30 minutes to about 8 hours, about 70 minutes to about 8 hours, about 80 minutes to about 8 hours, about 90 minutes to about 8 hours, or about 100 minutes to about 8 hours, including any range therebetween.

In some embodiments, a composition according to the present invention comprises a useful positron emitting isotope. In some embodiments, a suitable radiophore is prepared using the fluorine isotope 18F. Other useful positron-emitting isotopes may also be employed, such as 34Cl, 45Ti, 51Mn, 61Cu, 63Zn, 82Rb, 68Ga, 66Ga, 11C, 13N, 15O, and 18F. In one illustrative embodiment, the radioisotope is selected from 64Cu, 68Ga, 66Ga, and 18F. Factors that may be included during selection of a suitable isotope include sufficient half-life of the positron-emitting isotope to permit preparation of a diagnostic composition in a pharmaceutically acceptable carrier prior to administration to the patient, and sufficient remaining half-life to yield sufficient activity to permit extra-corporeal measurement by a PET scan. Further, a suitable isotope should have a sufficiently short half-life to limit patient exposure to unnecessary radiation. In an illustrative embodiment, 18F, having a half-life of 110 minutes, provides adequate time for preparation of the diagnostic composition, as well as an acceptable deterioration rate. Further, on decay 18F is converted to 180.

### Definitions

As used herein, the term “alkyl” describes an aliphatic hydrocarbon including straight chain and branched chain groups. In some embodiments, the alkyl group has 4 to 100 carbon atoms, and in some embodiments, 4-40 carbon atoms. Whenever a numerical range; e.g., “4-100”, is stated herein, it implies that the group, in this case the alkyl group, may contain 4 carbon atom, 5 carbon atoms, 6 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)2-R′ group, where R′ is as defined herein.

A “carbamyl” or “carbamate” group describes an —OC(═O)—NR′R″ group, where R′ is as defined herein and R″ is as defined for R′.

A “nitro” group refers to a —NO2 group.

A “cyano” or “nitrile” group refers to a —C═N group.

As used herein, the term “azide” refers to a —N3 group.

The term “sulfonamide” refers to a —S(═O)2-NR′R″ group, with R′ and R″ as defined herein.

The term “phosphonyl” or “phosphonate” describes an —O—P(═O)(OR′)2 group, with R′ as defined hereinabove.

The term “phosphinyl” describes a —PR′R″ group, with R′ and R″ as defined hereinabove.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

General:

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1A: Preparation of Hydrophobic Corroles According to Embodiments of the Invention

The hydrophobic corroles according to formula I listed in table 1 were prepared.

TABLE 1

| Corrole designation | M | R₉-R₁₁ | R₁-R₈ |
|---|---|---|---|
| Corrole 1 | H₃ | C₆F₅ | H |
| Corrole 1-Sb | Sb | C₆F₅ | H |
| Corrole 1-Sn | Sn | C₆F₅ | H |
| Corrole 1-Al | Al | C₆F₅ | H |
| Corrole 1-Ga | Ga | C₆F₅ | H |
| Corrole 1-Mn | Mn | C₆F₅ | H |
| Corrole 1-Gd | Gd | C₆F₅ | H |
| Corrole 1-Fe | Fe | C₆F₅ | H |
| Corrole 1-Au | Au | C₆F₅ | H |
| Corrole 1-P | P | C₆F₅ | H |
| Corrole 2-Al | Al | C₆F₅ | R₁-R₄ = I |
| | | | R₅-R₈ = H |
| Corrole 2-Au | Au | C₆F₅ | R₁-R₄ = I |
| | | | R₅-R₈ = H |
| Corrole 3 | H₃ | C₆F₄NH₂ | H |
| Corrole 4-Ga | Ga | C₆F₅ | R₁ and R₃ = SO₃H |
| | | | R₂, R₄-R₈ = H |
| Corrole 5 | H₃ | CF₃ | H |
| Corrole 5-Ga | Ga | CF₃ | H |
| Corrole 5-P | P | CF₃ | H |
| Corrole 5-Mn | Mn | CF₃ | H |

Corrole 4-Ga was prepared as a control.

### Example 1B: Preparation of Corrole 1

Corrole 1, having a compound name 5,10,15-tris(pentafluorophenyl)corrole, was prepared according to U.S. Pat. No. 6,541,628, incorporated herein by reference. A solid absorbent support (florisil, silica or alumina) (0.5 g) was mixed in a 50 mL flask with a 2 mL CH2Cl2 solution of 0.31 mL (2.5 mmol) of 2,3,4,5,6-pentafluorobenzaldehyde and 0.17 mL pyrrole (2.5 mmol), and the CH2Cl2 solvent was substantially removed through distillation at normal pressure. The condenser was removed and the solid mixture was heated to 100° C., upon which the color changed to black within 5-10 min. After heating for 4 h, the solid absorbent support was washed with 50 mL CH2Cl2, 0.25 g (1.1 mmol) 2,3-Dichloro-5,6-dicyano-1,4-benzoquinone (DDQ) was added, and the resulting corrole product was purified by chromatography on silica gel with hexane: CH2Cl2 (9:1) as eluent. The isolated chemical yield of corrole 1 was 11%.

### Example 1C: Preparation of Manganese Corrole 1-Mn

A mixture of 20 mg (25 micromole, μm) of 5,10,15-tri(2,3,4,5,6-pentafluorophenyl)corrole (corrole 1) and manganese (II) acetate tetrahydate (0.3 mmol) in 5 mL of freshly distilled DMF was heated to reflux for 1 h. After evaporation of the solvent the mixture was dissolved in CH2Cl2 and washed with 10% HCl. The product was recrystallized from CH2Cl2:hexane to provide the product in quantitative yield. Recrystallization from EtOH/water afforded 1-Mn as a green solid.

### Example 1D: Preparation of Gold Corrole 1-Au

Corrole 1 (20 mg, 0.025 mmol) was added to a solution of gold acetate (25 mg, 0.067 mmol) in pyridine under N2 atmosphere. Monitoring of the reaction was carried by thin layer chromatography (TLC) using 1:5 ratio of dicholoromethane (DCM):Hexane. After one hour the reaction was completed and the solvent was evaporated under reduced pressure without heating. The product was purified on silica-gel chromatography (silica gel, 1:1 Hexanes:DCM as eluent) in 12% yield. The identity of the compound was confirmed using NMR and mass spectroscopy.

### Example 1E: Preparation of Gold Corrole 2-Au

Corrole 1 (20 mg, 0.025 mmol) was added to a solution of gold acetate (25 mg, 0.067 mmol) and N-Iodosuccinimide (46 mg, 0.35 mmol) in pyridine. Monitoring of the reaction was carried by TLC (1:1 DCM:Hexane). After two hours the reaction was completed and the solvent was evaporated under reduced pressure without heating. The product was purified on silica-gel chromatography (silica gel, 1:1 Hexanes:DCM as eluent) in 33% yield. The identity of the compound was confirmed using NMR and mass spectroscopy.

### Example 1F: Preparation of Corrole 3

5,10,15-tris(pentafluorophenyl)corrole (20 mg, 0.025 mmol) was added to a solution of sodium amide (6 mg, 0.15 mmol) in dry tetrahydrofuran (THF) and stirred in a flask under inert and dry conditions overnight. The flask was moved to an ice bath and the reaction solution was quenched by addition of ice. Solvent was evaporated under reduced pressure and the product was purified on silica-gel chromatography (silica gel, 1:1 Hexanes:DCM as eluent) in 41% yield.

### Example 1G: Preparation of 1-Sb (Bis-Pyridine-Coordinated Product)

An about 3-fold excess of SbCl3 (86 mg, 0.38 mmol) was added to a pyridine solution (5 mL) of corrole 1 (100 mg, 0.126 mmol) in one portion. TLC examinations (silica gel, n-hexane/CH2Cl2)2:1) revealed that the starting material was fully consumed within 10 min at 100° C. Solvent evaporation and crystallization from CH2C2/pyridine/n-heptane mixtures yielded 109 mg (95%) of the title compound.

### Example 1H: Preparation of 1-Sn (Chlorine-Coordinated Product)

A solution of Corrole 1 (25 mg, 31.4 mmol) and SnCl2.2H2O (100 mg, 0.44 mmol) in DMF (3 mL) was heated to reflux for 30 minutes. The solvent was evaporated in a vacuum, and the residue was dissolved in CH2Cl2 and filtered. The filtrate was washed with concentrated HCl solution and evaporated. After recrystallization from hexane/CH2Cl2, the compound was obtained as dark violet crystals in 85% yield (25.3 mg, 26.7 mmol).

### Example 11: Preparation of 1-A1 (Pyridine Coordinated Product)

A 0.1 ml volume (200 μmol) of a 2 M AlMe3/toluene solution was added to a solution of 40 mg (50.3 μmol) of Corrole 1 in 4 ml toluene at room temperature under Argon. After 5 min, the reaction mixture was cooled by an ice bath and 0.1 ml of water was added to degrade excess AlMe3. After 3 addition of 1 ml pyridine, the reaction mixture was filtered and evaporated to dryness under vacuum. The title compound was obtained quantitatively and 40 mg (41 μmol, 81% yield) were obtained after recrystallization from n-hexane/CH2Cl2.

### Example 1J: Preparation of 1-Ga (pyridine coordinated product)

A solution of Corrole 1 in pyridine (15 ml) was added in large excess of flame-dried GaCl3 and the reaction mixture was heated to reflux for 1 hour under argon, followed by evaporation of the solvent. Excess inorganic salts were removed by flash chromatography (silica, n-hexane:Ch2Cl2:pyridine in a ratio of 100:30:0.5, followed by recrystallization ofrom Ch2Cl2 and n-heptane in the presence of a few drops of pyridine to give 45 mg (76% yield) of purple crystals.

### Example 1K: Preparation of 1-Fe (Pyridine Coordinated Product)

Corrole 1 (80 mg, 0.1 mmol) was dissolved in dry DMF (30 mL) under N2, and the addition of FeCl2 (0.25 g, 2 mmol). The mixture was heated to reflux, and the process was followed by TLC (silica, n-hexane/CH2Cl2 2:1). After completing the iron insertion, the reaction mixture was cooled to 25° C., and the solvent was evaporated. The resulting solid material was dissolved in diethyl ether, and chromatography over a short column (10 cm long, 2 cm diameter, with silica gel and diethylether as an eluent) was performed. The product, which was obtained after evaporation of the diethyl ether, was the diethyl ether-coordinated product. The title compound was obtained by recrystallization of the diethyl ether-coordinated product from diethyl ether/n-heptane mixtures in the presence of pyridine. The yield was in the range of 90-95% for pure crystalline materials.

### Example 1M: Preparation of 1-P ((OH)2-Coordinated Product)

POCl3 (400 mL, 4.3 mmol) was added in one portion to a boiling solution of compound 1 (40 mg, 50.2 mmol) in pyridine (4 mL) under N2. After 30 minutes of heating under reflux the solvent was evaporated, and the residue was dissolved in CH2Cl2 and filtered. The filtrate was washed with HCl (0.1 m), dried, and evaporated. The residue was passed through a column of silica with CH2Cl2 as eluent to remove excess of inorganic salts. After recrystallization from hexane/CH2Cl2, the title compound was obtained as red crystals in 87% yield (37.5 mg, 43.7 mmol).

### Example 2A: Synthesis of Corrole-Albumin Nanoparticles

Ten millimolar (mM) of a representative corrole according to formula [I] were dissolved in 400 microliters (μl) of dimethyl sulfoxide (DMSO) and were added dropwise at a rate of 0.04 milliliters (ml) per minute (min) into 3.6 ml of 1 mM of bovine serum albumin (BSA) or human serum albumin (HSA) in phosphate buffered saline (PBS) which has been adjusted to a pH of 9 by addition of sodium hydroxide. The mixture was then stirred at 500 rpm in a 5 degrees Celsius (° C.) water bath and incubated for 30 min at 5° C. The mixture was then transferred to dialysis tubing for 24 hour (h) dialysis in 1 liter (L) of pure water. Dialysis tubing used ere Regenerated Cellulose from Spectrum Labs having a molecular weight cutoff of 12-14 kilodaltons (kD). Dialysis tubings were prepared by treating vigorously with Ethylenediaminetetraacetic (EDTA) as follows: Tubing was immersed into 1 L of 2% sodium bicarbonate/1 mM EDTA in a 2 L glass beaker. Tubing was rinsed thoroughly with double distilled H2O. 50% ethanol/1 mM EDTA as added, and tubing was immersed and submerged completely. Tubing was rinsed before use. After dialysis, the corrole-albumin conjugate product was freeze dried for 24 h at −50° C. in a lyophilizer to dryness.

Dried product of the conjugates may be re-suspended in 4 mL PBS in order to theoretically yield a 1 mM corrole solution.

Running of the corrole samples (1-Sb, 1-Al, 1-Ga, 1-Mn, 1-Fe and 1-Sn) in PBS in a size exclusion chromatography column (S300 Sepharose gel column, using an eluent of PBS and a flow rate of 0.5 mL/min) yields a single signal by reading at 420 nanometers (nm) indicative of the corrole at a retention time of 15 min only, whereas the reading at the 280 nm for albumin shows a signal at the retention time of 15 min and at 35 min. This result indicates that the above process prepared corrole-albumin conjugates that elute at 15 min, as well as an excess of non-conjugated albumin, as indicated by the peak at 35 min.

The lyophilized conjugate product consists of a mixture of dry albumin and nanoparticles ranging in size of from about 10 nanometers in size to about 100 nm. The lyophilized product is stable for months under cooled storage. The re-suspended product is stable in solution for a few weeks. Transmission electron micrographs of the product were taken, and particle size was estimated based on the micrographs.

### Example 2B: Additional Synthesis of Corrole-Albumin Nanoparticles

Corrole-albumin conjugate is synthesized by dropwise addition (0.04 mL/min) of 400 μl of 10 mM of a representative corrole according to formula [I] dissolved in DMSO into 3.6 ml of 1 mM BSA or HSA in PBS (adjusted to pH 9 by sodium hydroxide) stirred at 500 revolutions per min (rpm) in a 5° C. water bath. The mixture is then incubated for 30 min at 5° C., followed by centrifugation of the product at 5000 g for 30 min. The solvent that contains unreacted BSA and DMSO is removed and the pellet is re-suspended with PBS. The product is then freeze dried for 24 h at −50° C. in a lyophilizer to dryness in order to achieve dry stable product or the re-suspended product is used. The thus obtained powder consists of nanoparticles with almost no free albumin, as confirmed by HPLC and native gel electrophoresis.

### Example 2C: Confirmation of Conjugation Between Corrole and Albumin Using Circular Dichroism and Complexes to the VLDL Fraction of Human Serum

Circular dichroism (CD) was used, employing light from the visible part of the spectrum to reflect corrole-albumin nanoparticles prepared according to example 2A.

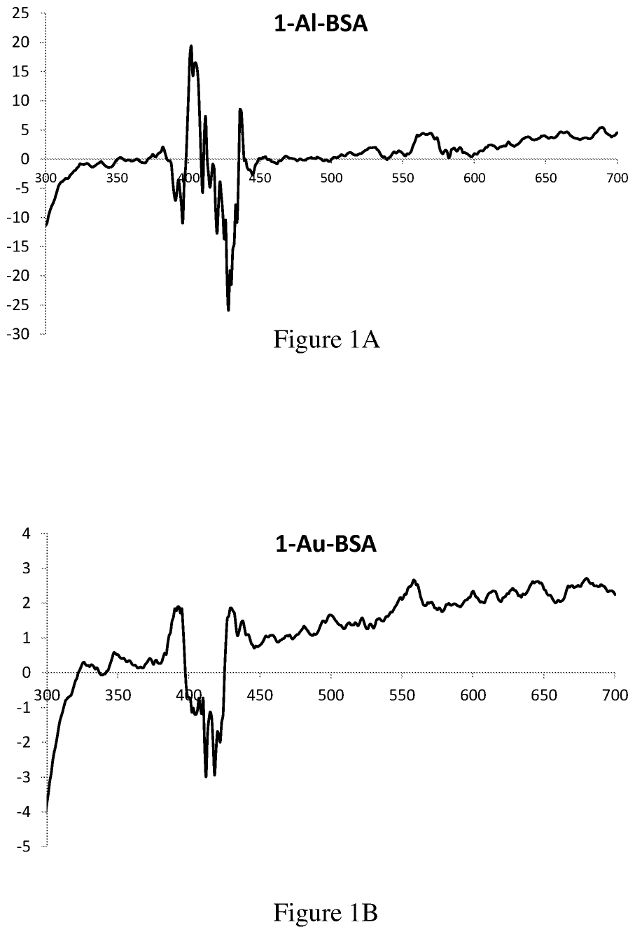

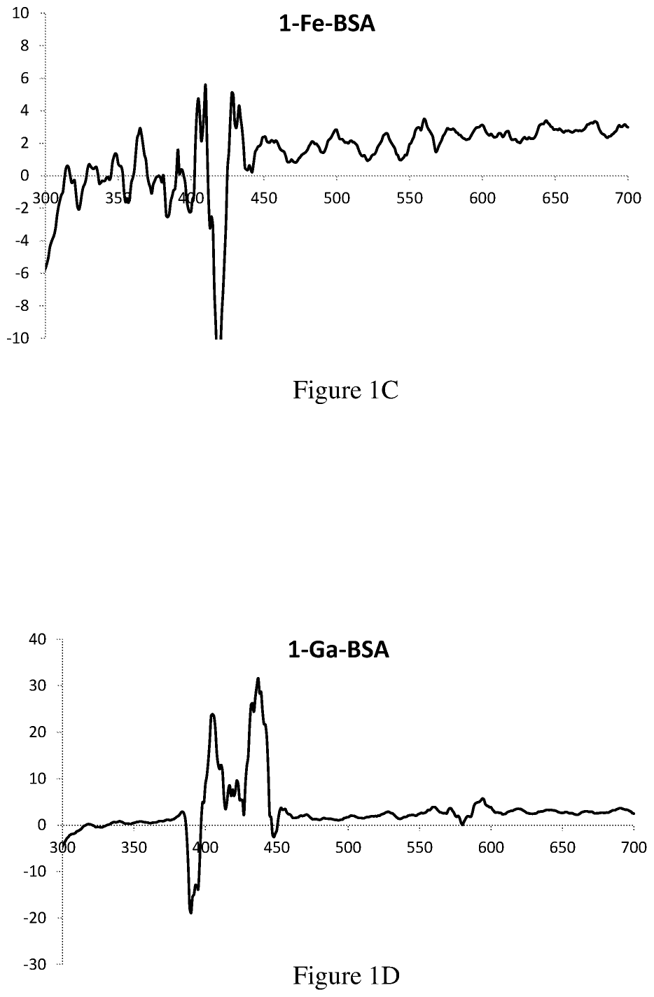

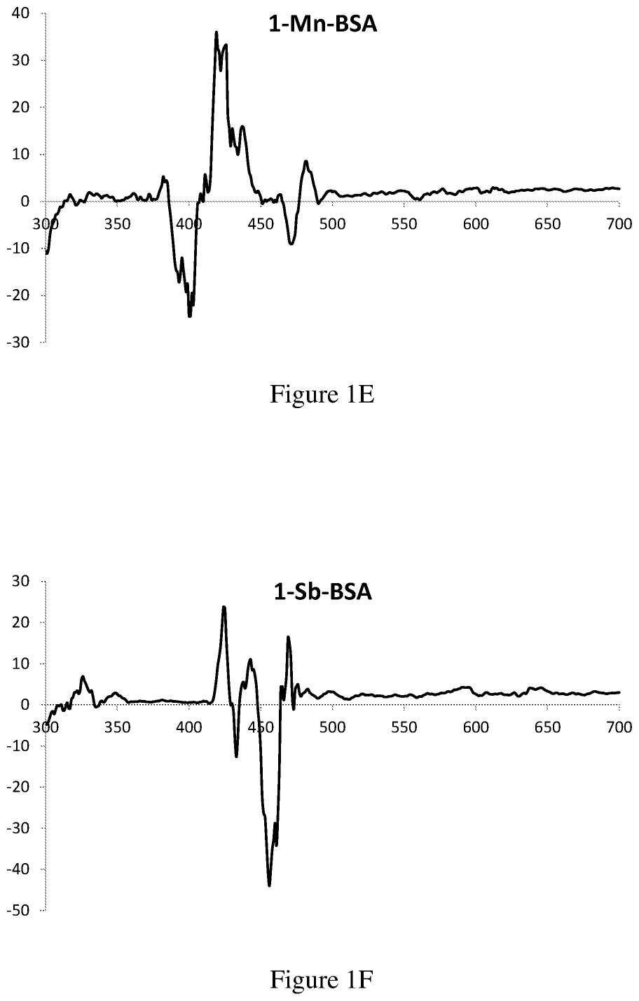

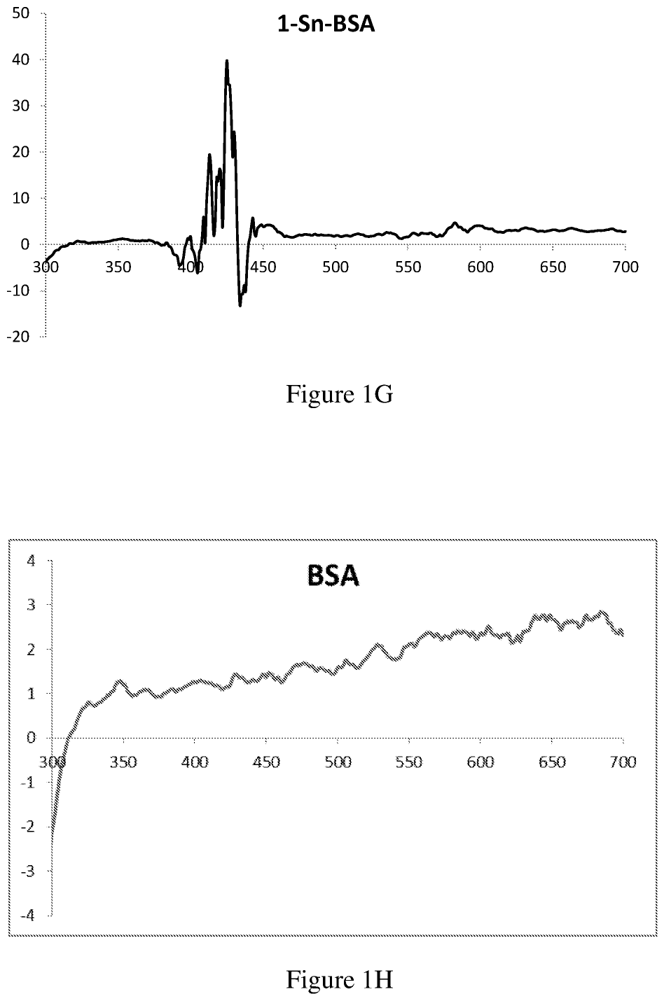

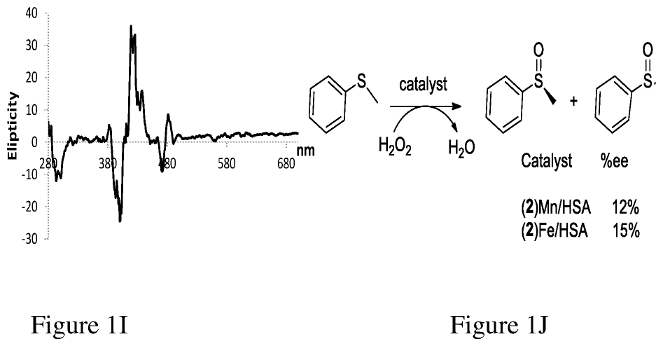

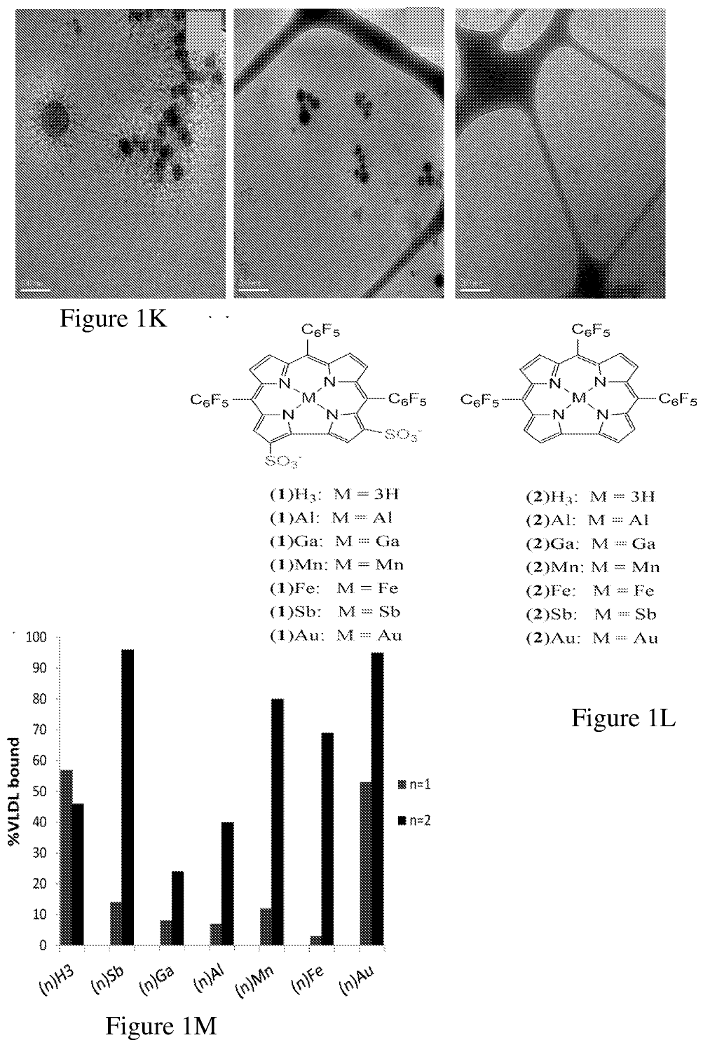

The corrole-albumin nanoparticles comprising the following metals were analyzed for chirality: 1-Al, 1-Au, 1-Fe, 1-Ga, 1-Mn, 1-Sb and 1-Sn. In addition, bovine serum albumin (BSA, alone without corrole, as a control) was also tested. The chromatograms showing CD are depicted in FIGS. 1A-1G, and for BSA alone, in FIG. 1H. The chromatograms show chirality of the corrole-albumin conjugates which may be attributed to the conjugation of the corrole with the protein. This was not evident in the control comprising albumin without corrole, as shown in FIG. 1H.

Additional information about NP constitution was obtained from additional CD analyses, deployment as asymmetric catalysts (FIG. 1I), and mass spectroscopy. Strong exciton CD coupling in the visible region corresponding to absorption of non-chiral (2)Mn is consistent with its intimate association within the chiral environment of albumin. Close association of metallocorroles with the chiral protein also was indicated by modest enantioselectivity in the H₂O₂ oxidation of thioanisole using albumin NP catalysts containing either (2)Mn or (2)Fe. It was unable to extract (2)Al into the organic phase following prolonged (24-h) stirring of (2)Al-based NPs in a PBS/CH2Cl2 solvent system. However, denaturation of the NP-bound protein with 2% Triton-X led to solubilization of (2)Al in the organic phase. Spectroscopic analysis of recovered corrole revealed no structural changes in the macrocyclic periphery (1H NMR, β-pyrrole CH protons) or to the C6F5 groups (19F NMR), thereby indicating that tight non-covalent association of the corrole NP with albumin occurred. Examination of NPs by MALDI MS provided additional evidence for noncovalent binding, as only signals corresponding separately to albumin and (2)Ga were observed.

As very low density lipoprotein (VLDL) is the primary carrier of lipophilic compounds, and it seems that enhanced VLDL loading could be attained by increasing corrole lipophilicity. Hence, another focus was turned to the much more lipophilic (2)M metallocorroles, where it was found that VLDL affinity was significantly enhanced, ranging from 24% for (2)Ga to almost 100% for (2)Sb and (2)Au. These experiments highlighted the solubility problem, as especially evident in 10% DMSO/PBS solutions: precipitation was observed at or higher than 100 □M corrole; and NPs formed in the 10-1 □M corrole range. It was further found that even the much more water-soluble (1)Ga forms NPs when combined with cancer-targeting proteins. Next, conjugating lipophilic metallocorrole [(2)M] NPs with native proteins was set out in the hope that these assemblies would be soluble in aqueous media. The first proteins examined were albumins (both bovine, BSA, and human, HSA), because of their abundance, stability and high solubility. FIG. 1L presents molecular structures of the investigated corroles and FIG. 1M shows the selectivity of (1)M (red bars) and (2)M (black bars) complexes to the VLDL fraction of human serum.

### Example 2D: Analysis of 1-A1 Albumin-Corrole Conjugate by Removal of Albumin

1-A1 was conjugated to BSA to form nanoparticles according to 2A and was extracted in PBS. Particles were treated with the non-ionic surfactant Triton-X (polyethylene glycol p-(1,1,3,3-tetramethylbutyl)-phenyl ether, octyl phenol ethoxylate,) 2% in order to dissociate the albumin from the corrole. Triton-X allowed for extraction of the corrole from water into dichloroethane (DCM). After mild mixing of a test tube comprising Triton-X and DCM, a separation between the aqueous and DCM layers was visible after 4 minutes of rest. The DCM fraction was poured onto a dry silica column using DCM only as an eluent. The Al corrole remained on the top of the column, thus allowing to remove any other extracted material. After a few washes with pure DCM, DCM with few drops of pyridine was added to the column for the elution of the aluminum corrole. The eluent was evaporated under vacuum and re-dissolved in C6D6 for NMR measurements. The spectra confirmed that there was no structural change of the corrole during formation of the albumin-corrole conjugate or subsequent removal of albumin.

### Example 2E: Characterization of Particle Size of Corrole-Albumin Conjugates

Cryo-Tunneling electron microscopic images were taken of 1-Au BSA nanoparticles after isolation by centrifugation. In liquid, nanoparticles appear as solubilized nano-spheres the size of about 100 nm or less, each with many string like extensions from the nano-sphere. In dry conditions, the nanospheres may aggregate to form larger aggregates, as shown by scanning electron microscope to particles having a size of about 200 nm.

FIG. 1K shows Cryo-TEM images of isolated (2)Au/BSA (denoted as “b” and “c”, at two different magnifications) and of similarly treated BSA but without corrole (denoted as “d”).

### Example 2F: Attempted Formation of Nanoparticles Using Other Hydrophobic Compounds

The formation of water-soluble nanoparticles comprising albumin were attempted with the hydrophobic compounds Au-TPP and doxorubicin. The synthesis of [Au(TPP)]Cl was conducted under a nitrogen atmosphere using standard Schlenk technique. To a mixture of K[AuIIICl4] (0.5 mM) and sodium acetate (2.5 mM) heated at 80° C. in acetic acid (20 mL), a solution of free tetraphenylporphyrin (TPP, 0.4 mM) in 10 mL acetic acid was added, and the mixture was refluxed for 2 hr. After removal of solvent by vacuum, the residue was dissolved in CH2Cl2, and was washed twice with water to remove any unreacted K[AuIIICl4] and sodium acetate, followed by chromatographic purification using neutral 90-alumina with CH2Cl2/MeOH as an eluant. The product gold(III) porphyrin was obtained in 70% yield after ion exchange using aqueous LiCl.

1-Au, Au-TPP and doxorubicin were each dissolved in DMSO to a concentration of 1 mM. Each of them was added to a 100 □M BSA solution in PBS up to the point that the mixture contained 100 □M of the compound and 10% (v/v) DMSO. Samples were incubated at 4° C. for 30 min, after which samples were centrifuged at 20,000 g for 30 min. This set of experiments revealed that the lipophilic 1-Au induces the spontaneous formation of nanoparticles while the same process performed with Au-TPP and with doxorubicin does not form nanoparticles. After centrifugation of 1-Au, a pellet is formed, indicating that there is a solid dispersion. Au-TPP and doxorubicin formed a solution in which a pellet is not formed after centrifugation at the same conditions.

### Example 2G: Solubility of Corrole-Albumin Nanoparticles in Water

Solubility of corrole-albumin nanoparticles in PBS having a pH of 7.2 was determined by dissolving BSA conjugates of the corroles 1-Sn, 1-Fe, 1-Mn and 1-A1 in PBS. The solubility for all of the conjugates was determined to be readily soluble (40 mg/ml). The corroles, in non-conjugated form all are non-water soluble, with a solubility of less than 10-7 mg/ml.

### Example 2H: Characterization Methods

SEM Analysis:

100 μL of NP samples were placed on silicon wafers and left inside a chemical hood to fully dry. Silicon wafers were then taken to be coated with gold using Polaron Sputter coater. Samples were measured by TESCAN (Vega-II) Scanning Electron Microscopy supported by TESCAN software system.

AFM analysis:

100 μL of NP samples were placed on silicon wafers and left inside a chemical hood to fully dry. Samples were than measured using a Veeco (Dimension 3100) Atomic Force Microscope (AFM) operated by a NanoScope IIa Controller.

DLS and Nanosight NS300 systems:

Samples were diluted by a factor of 10,000 in PBS. They were measured using the Nanosight NS300 or the PSS Nicomp 380 DLS-ZLS Analyzer in accordance with manufacturer's instructions.

Cryo-TEM Analysis:

Samples were prepared to be at a mass percentage of 1% particles in PBS and the Cryo-TEM specimens were prepared in a controlled environment vitrification system (CEVS). Cryogenic transmission electron microscopy (cryo-TEM) imaging was performed either by a Phillips CM120 or a FEI Talos 200C, FEG-equipped cryo-dedicated high-resolution transmission electron microscope (TEM and STEM), operated at an accelerating voltage of 120 kV. Specimens were transferred into an Oxford Conn.-3500 cryo-holder (Philips) or a Gatan 626DH (FEI) cryo-holder, and equilibrated below −178° C. Specimens were examined using a low-dose imaging procedure to minimize electron-beam radiation damage. Images were recorded digitally by a Gatan Multiscan 791 cooled CCD camera (Philips CM 120), or a Gatan US 1000 high-resolution CCD camera (Tecnai T12 G2), using DigitalMicrograph software.

HPLC:

HPLC analysis was performed using a MERCK HITACHI HPLC system with a diode array detector supported with HPLC Chromaster Driver for Waters® Empower™3 Software. 10 μL of each sample was injected using the auto sampler. Size exclusion chromatography was done on a Superose™ 6 10/300 GL gel column and on a Sephadex™ 200 10/300 GL column (as noted), with 0.5 mL/min eluting rate and sterilized PBS (Sigma, sterile-filtered. isotonic, pH 7.2) as eluent.

Human Cancer Cell Lines:

One cell line from the NCI60 cell panel was used in this study: DU-145 (prostate cancer). Cells were grown in EMEM cell culture medium (ATCC) containing 2 mM L-glutamine, supplemented with 10% FBS (Biological Industries), and maintained at 37° C. under 5% CO2 in a humidified incubator.

FACS Analysis, General Procedure:

Cells (DU-145) were seeded in 6-well microtiter plates (5×104 cells/mL; 3 mL per well) 24 h before the addition of assigned compounds/albumin particles. At the time of drug treatment, stock solutions of compounds were diluted to 10-fold the desired final test concentrations with EMEM medium. Aliquots of 300 μL of these diluted solutions were added to the appropriate microtiter wells containing 2700 μL of medium, resulting in the required final drug concentrations (according to assigned concentration). The final concentration of DMSO (given that the compound required DMSO for solvation) in test culture was <1%. All cells were incubated in the dark throughout the 24-72 h exposure period and did not receive prolonged exposure to light. Following 24-72 h of exposure at 37° C., cells were re-suspended with trypsin into PBS pH 7.2 and pelleted at 3000 rpm for 5 min. Samples were washed under PBS×3 times. Samples were taken for measurement using a BD LSR-II Analyzer supported with BD FACSDiVa™ Software Version 6.1 for data analysis. Corrole fluorescence was measured using appropriate filters for excitation (405 nm, DAPI) and emission (Ds-RED).

Live Cell Imaging, General Procedure:

Cells (DU-145) were seeded in a 24-well microtiter plates with a glass optic bottom (5 ×104 cells/mL; 0.9 mL per well) 24 h before the addition of assigned compounds/NPs. At the time of drug treatment, stock solutions of compounds were diluted to 10-fold the desired final test concentrations with EMEM medium 1640. Aliquots of 100 μL of these diluted solutions were added to the appropriate microtiter wells containing 900 μL of medium, resulting in the required final drug concentrations (according to assigned concentration). Mitotracker Green (MTG), lysotracker green (LTG) and ertracker green (ETG) (Life Technologies, Rhenium, Jerusalem, Israel) were added in accordance with manufacturers instructions and were optimized for best assigned concentrations/incubation time and tagging results. The final concentration of DMSO (given that the compound required DMSO for solvation) in test culture was <1%. All cells were incubated in the dark throughout the 24 h exposure period and did not receive prolonged exposure to light until excitation by assign wavelengths. Following 24 h of exposure at 37° C., cells were treated with MTG (200 nM, 30 min), LTG (150 nM, 2 h) and ETG (2 μM, 2 h). Corrole uptake and organelle tagging was measured using a ×40 objective and a LSM700 confocal system supported with Zen software. Samples were excited at 488 nm for MTG, LTG and ETG detection (3%, 5% and 5% respectively); and at 405 nm (10%) for (2)Ga detection.

Separation of Corrole-Containing Serum:

500 μL of 200 μM of aqueous corrole solutions were added to 500 μL of human serum and co-incubated at room temperature for at least 15 minutes prior to further treatment. The solutions were filtered through a 0.22 μm filter and 50 μL were injected to a LaChrom Elite HPLC system fitted with a superose 6 10/300 GL (GE healthcare) gel filtration column and a photodiode array detector. The samples were eluted with PBS (pH 7.2) at a flow rate of 0.5 mL/minute. Chromatograms at 280 nm and at the λmax of each corrole (424, 433, 426, 420, 424, 420 and 426 nm for (½)H3, (½)Au, (½)Sb, (½)Mn, (½)Al, (½)Fe and (½)Ga respectively) were recorded. The electronic spectra were recorded at time frames where the eluted corrole-containing lipoproteins were maximal.

Statistical Analysis:

Data were expressed as the mean±S.E.M and were compared between experimental groups with the use of one-way analysis of variance followed by Tukey's post hoc test unless otherwise specified (Analyze-it software for Windows Excel, Leeds, UK). Probability values of p<0.05 or less were considered statistically significant.

NMR:

The ¹H NMR and 19F NMR spectra were recorded on Bruker AM 200 and AM 300, operating at 200 and 300 MHz for 1H and 188 MHz for 19F (on AM 200), respectively. Chemical shifts are reported in ppm relative to residual hydrogen atoms in the deuterated solvents: 7.24 for chloroform.

### Example 3: Synthesis of Corrole-Transferrin Nanoparticles

Transferrins are plasma proteins active in regulating amount of iron in blood plasma. Ten millimolar (mM) of a representative corrole according to formula [I] is dissolved in 400 microliters (μl) of dimethyl sulfoxide (DMSO) and is added dropwise at a rate of 0.04 milliliters (ml) per minute (min) into 3.6 ml of 1 mM of apo-transferrin in phosphate buffered saline (PBS) which has been adjusted to a pH of 9 by addition of sodium hydroxide. The mixture is then stirred at 500 rpm in a 5 degrees Celsius (° C.) water bath and incubated for 30 min at 5° C. The mixture is then transferred to dialysis tubing for 24 hour (h) dialysis in 1 liter (L) of pure water. Dialysis tubing used are Regenerated Cellulose from Spectrum Labs having a molecular weight cutoff of 12-14 kilodaltons (kD). Dialysis tubings were prepared by treating vigorously with Ethylenediaminetetraacetic (EDTA) as follows: Tubing is immersed into 1 L of 2% sodium bicarbonate/1 mM EDTA in a 2 L glass beaker. Tubing is rinsed thoroughly with double distilled H2O. 50% ethanol/1 mM EDTA is added, and tubing is immersed and submerged completely. Tubing is rinsed before use. After dialysis, the corrole-transferrin conjugate product is freeze dried for 24 h at −50° C. in a lyophilizer to dryness.

Dried product of the conjugates may be re-suspended in 4 mL PBS in order to theoretically yield a 1 mM corrole solution.

Running of the resulting corrole-transferrin conjugate samples (1-Sb, 1-Ga, 1-Au, 1-Fe, 1-Sn, 1-Mn, 1-A1 and compound 1) in PBS in a size exclusion chromatography column (S300 Sepharose gel column, using an eluent of PBS and a flow rate of 0.5 mL/min) yields a single signal by reading at 400-450 nanometers (nm) indicative of the corrole-containing material at a retention time of 16 min only, whereas the reading at the 280 nm for transferrin-containing material shows a signal at the retention time of 16 min and at 26 min. This indicates that the above process leads to corrole-transferrin conjugates that elute at 16 min, and an excess of non-conjugated transferrin, as indicated by the peak at 26 min.

The lyophilised conjugates consist of a mixture of dry transferrin and nanoparticles ranging in size of about <100 nm. Product is stable as solid for months under cooled storage. The re-suspended product is stable for a few weeks.

### Example 4A—Treatment of Cancer Using Photodynamic Therapy (PDT) with Corrole-Protein Conjugates

PDT is a process which is based on accumulation of photosensitizers (PS) in cancer cells, which upon irradiation by tissue-penetrating wavelengths activate molecular oxygen to either singlet oxygen or reduced oxygen. Both species are highly toxic and induce cell death. Certain corroles may act as photosensitizers and when they enter cells, may lead to cell death when irradiated by tissue-penetrating wavelengths.

Without being bound by theory, it is suggested that corrole-protein nanoparticles may comprise a corrole, when in circulation in a human body, may present in a state in which emission of radiation from the corrole is ineffective due to self-quenching in the nanoparticle. This hypothesis was supported by comparing the fluorescence of 1-Ga in its free and nanoparticulate states, noting a 2 order of magnitude intensity decrease in the latter relative to the former. Once the corrole-protein nanoparticles enter a target cell, the corroles become emissive (tested for the DU-145 cell line and 1-Ga/BSA nanoparticle) and when excited by radiation having tissue-penetrating wavelengths, emit radiation capable of killing cells (exposure of DU-145 cell line incubated with 1-Ga/BSA nanoparticle to light induced extensive cell death). As a result, there is little systemic photosensitivity and only cells that have internalized the corrole-protein conjugates become vulnerable to the PDT.

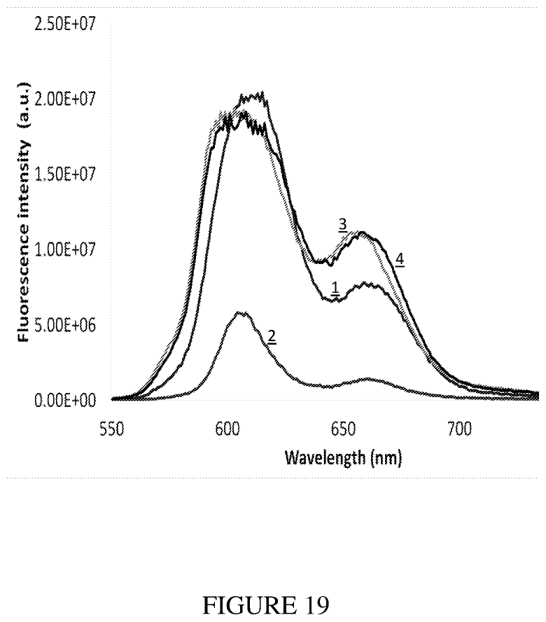

In additional exemplary procedures, compelling evidence for corrole aggregation within the assembly was obtained by comparing the emission spectra of (2)Ga-based NPs with virtually identical concentrations of the same corrole under conditions where no NPs were formed (FIG. 19). Utilizing the same amount of a DMSO stock solution of (2)Ga for dilution into either DMSO (blue trace) or BSA-containing PBS buffer (red trace), we found that fluorescence in aqueous media was strongly quenched. Full recovery of emission was observed upon treatment with Triton-X above its critical micelle concentration, in both the aqueous medium (green trace) and when the monomerized (2)Ga was extracted into an organic solvent (the black trace). Triton-X (green trace), and extraction of (2)Ga from the last solution into dichloromethane (black trace). Note that instrumental parameters were the same in all cases.

The selectivity to cancer cells has been demonstrated by the different uptake of albumin and transferrin based corrole nanoparticles by various cell lines. Cancer cells tend to allow entrance of nanoparticles and/or albumin, because of increased number of membrane receptors for these proteins relative to other cells.

Preferable corroles to be used for PDT are those that have low dark-toxicity (toxicity when not irradiated by tissue penetrating wavelengths) and high light-toxicity (toxicity when irradiated by tissue penetrating wavelengths). The identity of the wavelength of activation radiation and nature of the dark vs. light-induced cytotoxicity depends upon the identity of the corrole chelated element (M in Scheme 1) and the substituents on the corrole periphery (R1-R8 in Scheme 1). In a preferred example, M=Al, R1-R4=I, R5-R8=H, R9-R11=C6F5, (known as 2-Al) as that complex has no dark toxicity, provides a long-lived triplet excited state required for the reaction with molecular oxygen, and strong absorbance at the tissue-penetrating wavelengths of >650 nm.

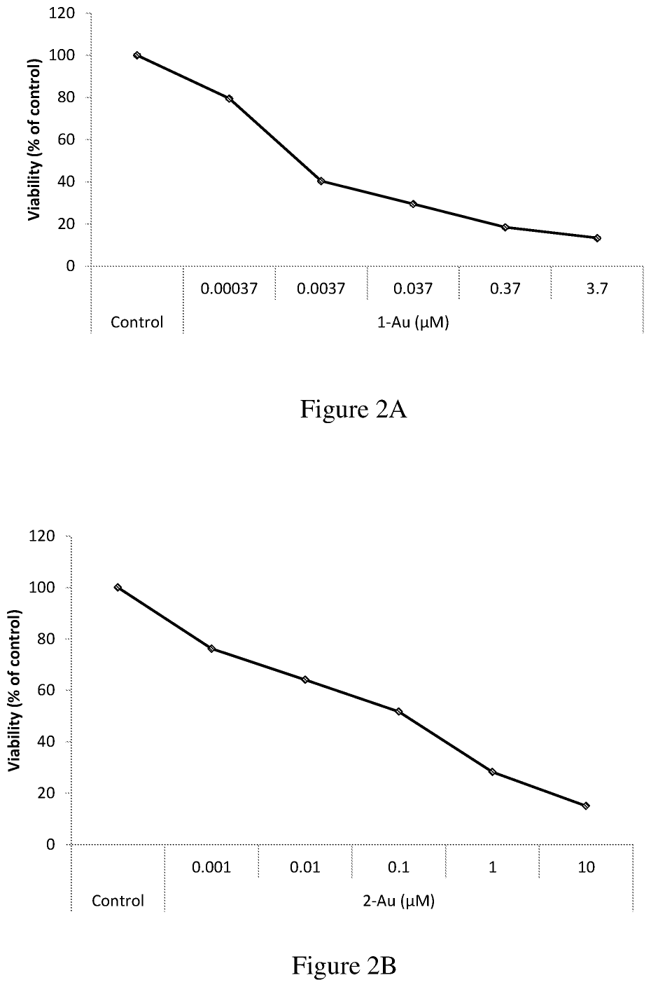

Cytotoxic effect of nanoparticles from corrole 1-Au-BSA and corrole 2-Au-BSA were tested in cells. Pancreatic cancer cells from the DU-145 cell line were pretreated with BSA control or with corrole—BSA nanoparticles. Cell viability was evaluated by an “MTT” assay 48 h later. The MTT assay is a colorimetric assay that measures cell metabolic activity using the tetrazolium dye MTT (3-(4,5-dimethylthiazol-2-yl)-2,5-diphenyltetrazolium bromide) and the formazan reduction product thereof, which has a purple color. Cells were plated and then incubated. 10 microliters of MTT was added and incubated for 2-4 hours until a purple precipitate was visible. After adding detergent and leaving in the dark for 2 hours, absorbance was recorded. IC50 values for 1-Au ranged in the single nanomolar concentration (<0.0037 μM) and for 2-Au in the hundreds nanomolar (0.1 μM). The results of cell viability at various concentrations using 2-Au-BSA and 1-Au-BSA are shown in FIGS. 2A and 2B. These results show toxicity in cancer cells of corrole-protein nanoparticles using a variety of corroles.

### Example 4B—Use of Corrole-Protein Nanoparticles as Contrast Agents for Magnetic Resonance Imaging (MRI)

Corrole-albumin nanoparticles comprising 1-Mn or 1-Gd corroles can be used as magnetic resonance imaging (MRI) contrast reagents. The T1 relaxation time of water of the aqueous solution of corrole-albumin nanoparticles, prepared from the manganese corrole 1-Mn and albumin was determined it to be faster by 30% than that of a solution of the albumin only. Cells, for example cancer cells may be incubated with the abovementioned corrole-albumin nanoparticles for MRI-based labeling.

Corrole-albumin nanoparticles comprising 1-Mn or 1-Gd corroles were dissolved in D20 and an inversion recovery assay was performed at 200 megahertz (MHz) to estimate of relaxivity induced by agent at 3 T standard MRI machines. T1 are given at highest concentration of 100 μM of TPFC-Mn and at the respective concentration of control protein. Estimated r1 value (indicative of the concentration vs. effectiveness of the contrast agent) was recovered from the slope of the relation 1/ΔT1=r1 [c contrast agent], as shown in table 2 below:

TABLE 2

| Sample | T1 (in seconds) | Estimated r1 |
|---|---|---|
| BSA only (control) | 4.6 | — |
| 1-Mn nanoparticle | 3.2 | 7.14 |

### Example 4C: Use of Corrole-Protein Nanoparticles for Inactivation of Biofilms by Photodynamic Inactivation (PDI)

It has been demonstrated that the principle of PDT may be also used for the photodynamic inactivation (PDI) of biofilms via their exposure to sunlight. Corroles that are water-soluble and are either positively- or negatively-charged were used for inactivation of mold fungi and green algae. Derivatives with positively-charged groups were more active, while lipophilic corroles could not be used due to their insolubility in water. The solubility of the corrole-protein nanoparticles shown above, allows the lipophilic corroles to be used for PDI. In a preferred example the corrole-protein are prepared by combing corrole 1-P with either albumin or transferrin.

### Example 4D: Use of Corrole-Protein Nanoparticles for Treatment of Disease Associated with Reactive Oxygen Species (ROS) or for Extending Lifespan

Oxidative stress, i.e., the imbalance between the minute amounts of reactive oxygen species (ROS) needed for many normal function and the activity of enzymes responsible for destruction of the excessive ROS that are highly toxic, is known to shorten lifespan and is associated with many disease mechanisms. Water soluble corroles have shown effect in treatment of atherosclerosis (the early stage leading to cardiovascular diseases), intracellular biosynthesis of cholesterol (the most established risk factor of cardiovascular diseases), diabetes complications, damage to the insulin-producing beta cells, and a variety of neurodegenerative diseases. Corrole-protein nanoparticles may be used to administer in patients in need thereof suffering from diseases associated with oxidative stress. In a preferred example, the iron corrole 1-Fe, which is capable to catalytically destroy ROS, conjugated to either albumin or transferrin is administered to the patient in need thereof. The easy solubility of such a conjugated corrole-protein may allow for its utilization in a medium that is consumed, optionally via the oral route, by the patient.

### Example 4E: Use of Corrole-Protein Nanoparticles for Asymmetric Catalysis

Transition metal-chelated corroles (M=Mn, Fe, Co, Cu) can be catalysts for a variety of atom- and group-transfer to organic substrate. The most common fashion for inducing enantioselectivity (production of a non-identical mixture of two enantiomers) is to covalently attach mono-chiral moieties to the complex, which requires multi-step synthesis and is a very non-flexible approach. The other option is to have the catalyst in a chiral environment, which in the case of the corrole-protein nanoparticles is provided by the protein. In the preferred example, the corrole-protein nanoparticles comprising 1-Mn with either albumin or transferrin may be used for oxidation of sulfides to sulfoxides by hydrogen peroxide. Exemplary use includes the sulfide precursors of the approved antiulcer drug (S)-omeprazole and the anti-narcolepsy drug Nuvigil (Armodafinil), whose chirality is based on an asymmetric sulfoxide moiety. Utilizing albumins from a large variety of sources (human, bovine, horse, etc.) allows for variation in the chiral environment.

### Example 4F: Use of Corrole-Protein Nanoparticles for Dye Sensitized Solar Cells (DSSC)

DSCC (also called Graetzel Cells) rely on the binding of photosensitizers (PS) to solid electrodes made from materials such as TiO2, which can be achieved by PS that have negatively charged head groups (—CO2- is most common). On the other hand, albumin has been shown to bind to TiO2 without any prior treatment. According to an embodiment of the invention, a TiO2 electrode is immersed into a solution of the corrole-protein nanoparticle comprising 1-A1 and albumin, and corrole protein nanoparticles bind to the electrode. After the binding, the electrode is washed by solvent and used in the DSCC in the usual fashion. This circumvents the need to synthesize PS with charged head groups and allows for flexibility in many terms, including the particular source of the albumin.

### Example 4G: Use of Corrole-Protein Nanoparticles for Optical Imaging of Cells, Such as Cancerous Cells

Incubation of the corrole-protein nanoparticles comprising a fluorescent corrole and either albumin or transferrin allows for optical imaging of cells, with varying intracellular distribution. The particular plasma protein used for the formation of the nanoparticles may induce selectivity to cancerous cells that overexpress (relative to normal cells) receptors of the particular protein. Specific-to-organ tumor imaging is achieved by one of the most widely characterized receptor-mediated transcytosis systems for the brain, based on targeting the transferrin receptor (TfR) that is highly expressed on endothelial cells of the blood-brain-barrier (BBB). Fluorescent corrole-protein nanoparticles may be used for this use. Example 4A addresses exemplary corrole-protein nanoparticles which may be used for this use as they fluoresce upon radiation at various wavelengths.

### Example 4H: Use of Corrole-Protein Nanoparticles for Sonodynamic Therapy (SDT)

SDT has been gaining increasing attention in recent years as a non-invasive method for the eradication of solid tumors in a site-directed manner. SDT involves sensitization of a tissue using relatively low-intensity ultrasound waves which excite a non-toxic sensitizing chemical, known as a sono-sensitizer. The ideal SDT agent will have intrinsic properties of low cytotoxicity, but high toxicity upon exposure to ultrasound. Without being bound by theory, it is suggested that the method of action of a sono-sensitizer is that ultrasound waves produce cavitation and the rapid collapse of microbubbles in the fluid, which may produce sonoluminescence and drive ultrasound induced chemical reactions upon which the sono-sensitizer becomes excited and initiates cytotoxic events. Therefore traits that are beneficial to PDT activity of a compound may be used for SDT as well, without the potential disadvantage of calibrating appropriate wavelength of absorption to fit for passage through hard tissue. In the case of nanoparticles of corroles, the low phototoxcicity is inherent to the self-quenching effect of the corroles present within the protein nanoparticles. Furthermore, such particles will have specificity towards malignant tissue, since malignant tissue displays porous capillaries which facilitate transfer of bigger structures (like nanoparticles) from the blood stream in to the malignant tissue. The aluminum corrole nanoparticles may be most beneficial to this application, because of their low inherent cytotoxicity.

Another possible therapeutic approach using corrole-protein nanoparticles is to combine PDT and SDT. Compounds that may lack effect as standard PDT agents due to improper absorbing wavelengths and/or the inability of external light to reach the target tumor are excited to the oxygen-activating excited state via sonication. The gallium-based nanoparticles may be most beneficial to this application, because of their low “dark” cytotoxicity on one hand and the effective intersystem crossing rate that is required for reaching the excited triplet state.

### Example 5: Detection of Whole Complex in Sera In Vivo

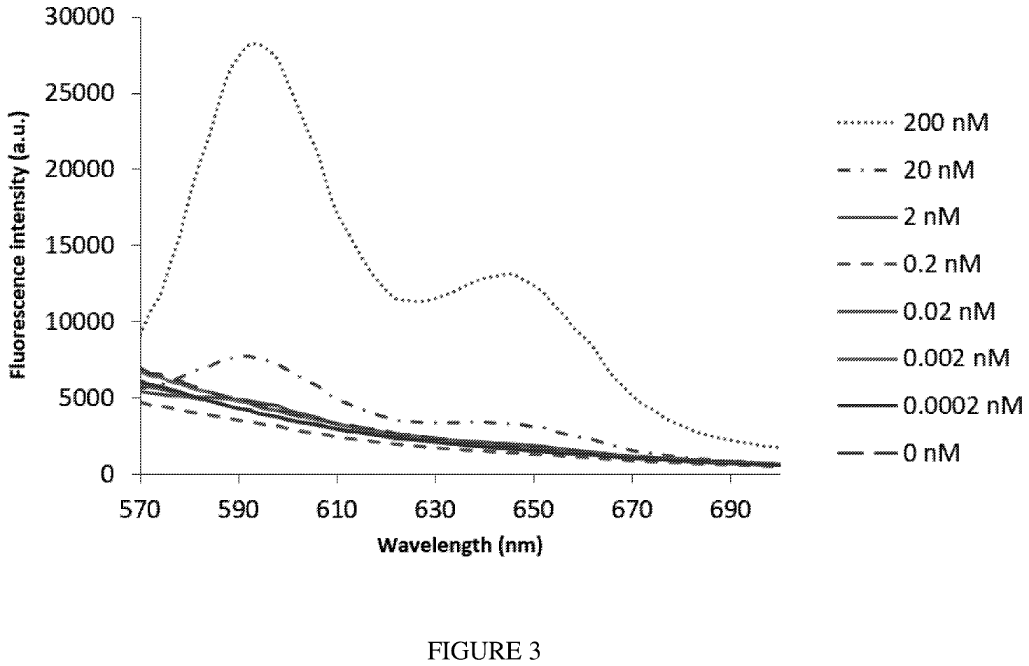

FIG. 3 shows the fluorescence detection limit in sera of Corrole 5-Ga at different concentrations.

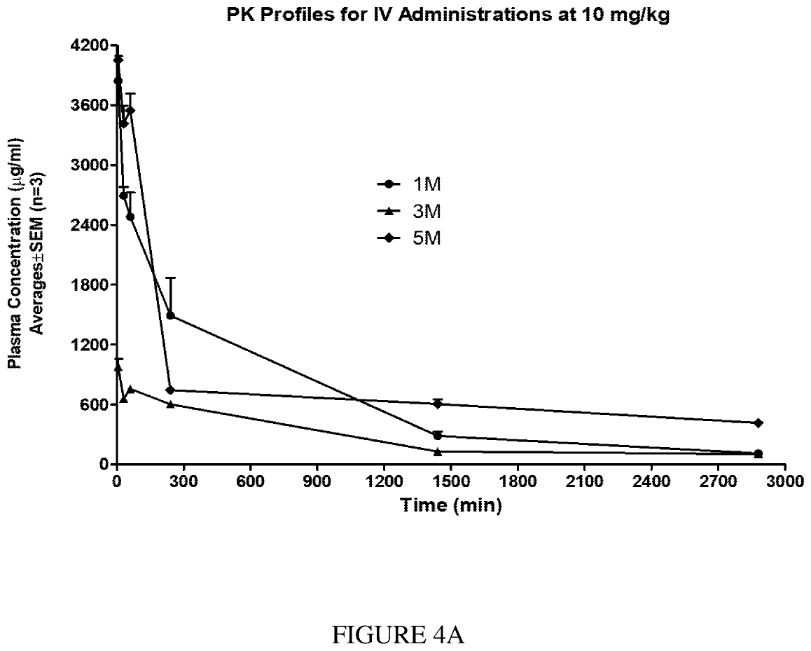

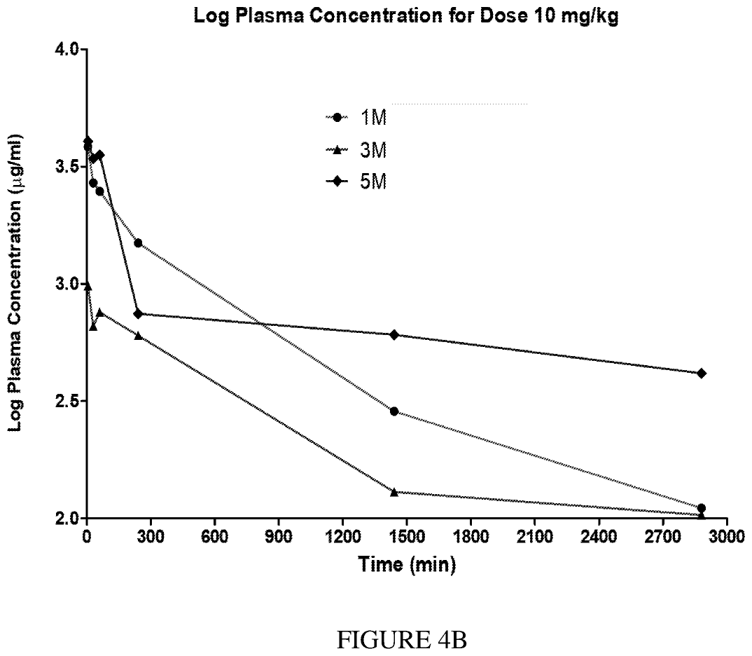

FIGS. 4A-B show the PK profiles of Corrole 4-Ga, Corrole 1-Ga and Corrole 5-Ga (1M, 3M and 5M respectively) at the 10 mg/kg dose both on a normal (FIG. 4A) and logarithmic scale (FIG. 4B).

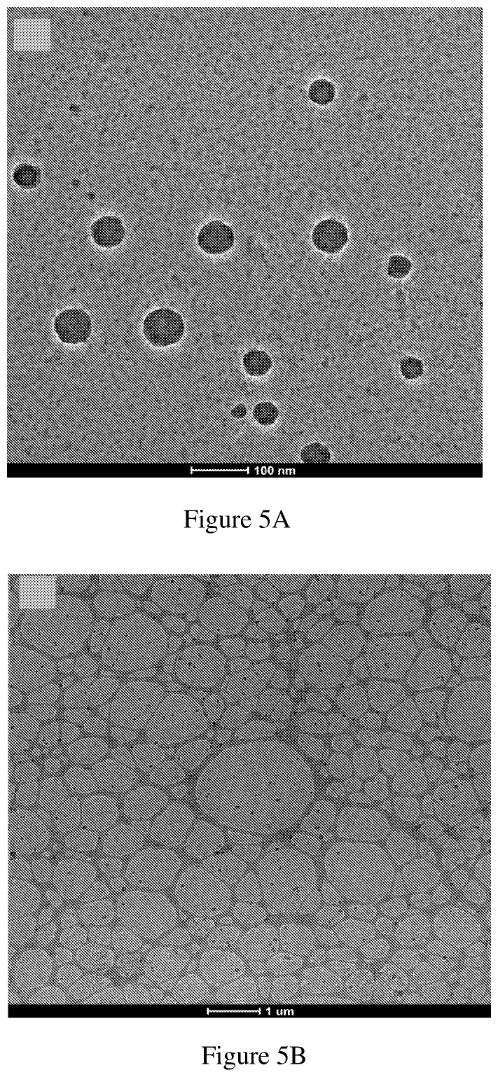

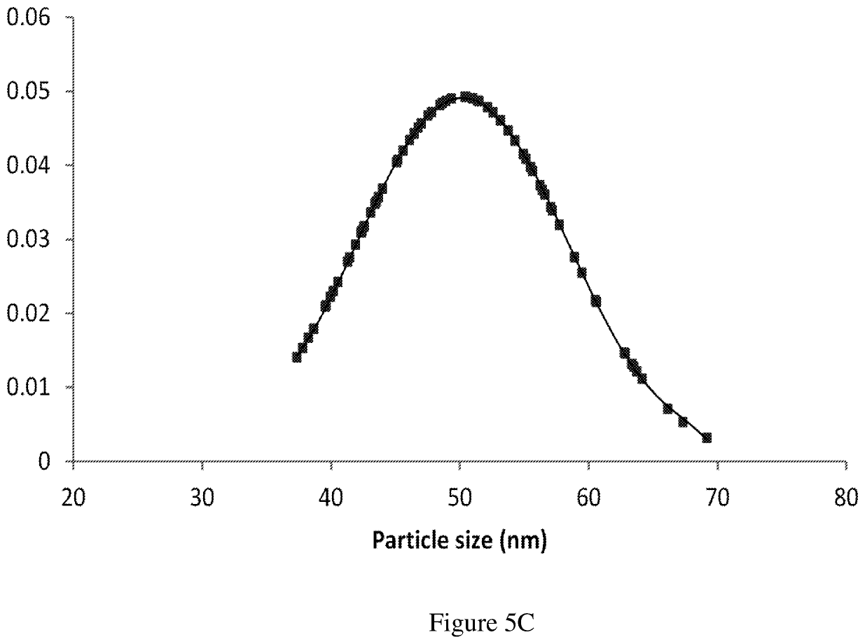

FIGS. 5A-B are representative cryo-TEM images of Corrole 5-Ga/HSA based NP's with high magnification (FIG. 5A) and lower magnification (FIG. 5B) to show homogeneity of structure (carbon greed is visible).

FIG. 5C is a graph showing the distribution analysis of particle size analyzed from 12 separate fields in two distinct samples of Corrole 5-Ga/HSA NP's. Particles were spherical in form with an estimated diameter of 50.18±8.1 nm.

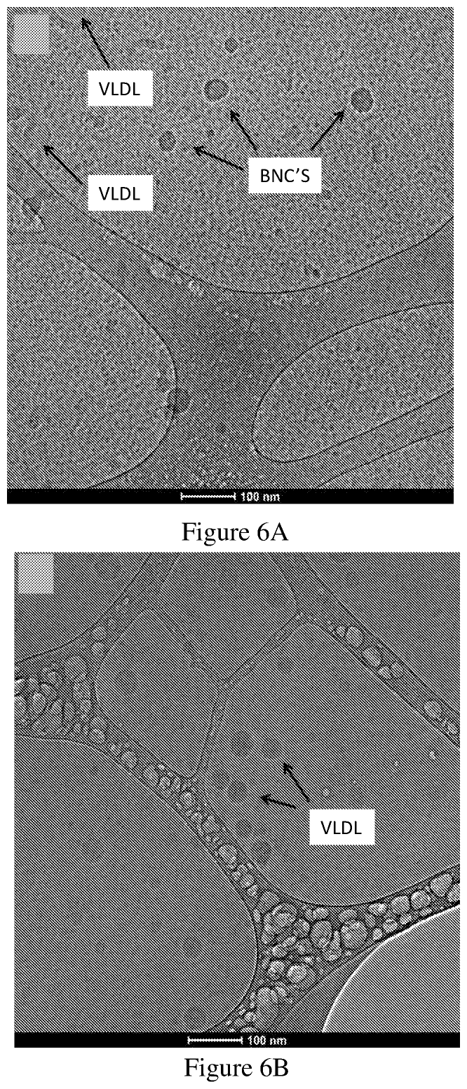

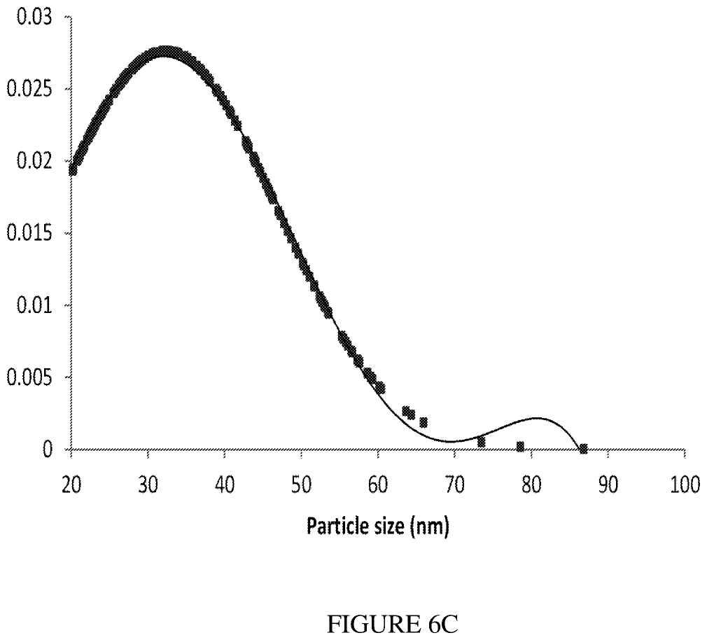

FIGS. 6A-B are representative cryo-TEM images of Samples of sera from mouse treated with Corrole 5-Ga/Human serum albumin (HSA) nanoparticles (NP's) (FIG. 6A) and vehicle at time points 30 minutes (FIG. 6B).

FIG. 6C is a graph showing the distribution analysis of particle size analyzed from 12 separate fields in two distinct samples of Corrole 5-Ga/HSA NP's treated mice.

The architecture of the particles is consistent with the original particles before administration (FIGS. 5A-C) with somewhat smaller sizes post administration. This indicates that the particles maintain their structure and are stable within circulation. Nanoparticular conjugates of corroles and albumin are stable upon circulation in-vivo 30 min post intravenous (i.v.) administration. This is evident in cryo-TEM images of serum samples collected 30 min post i.v. administered of 10 mg/Kg 5-Ga conjugated to HSA.

### Example 6: Calcium Homeostasis Perturbation

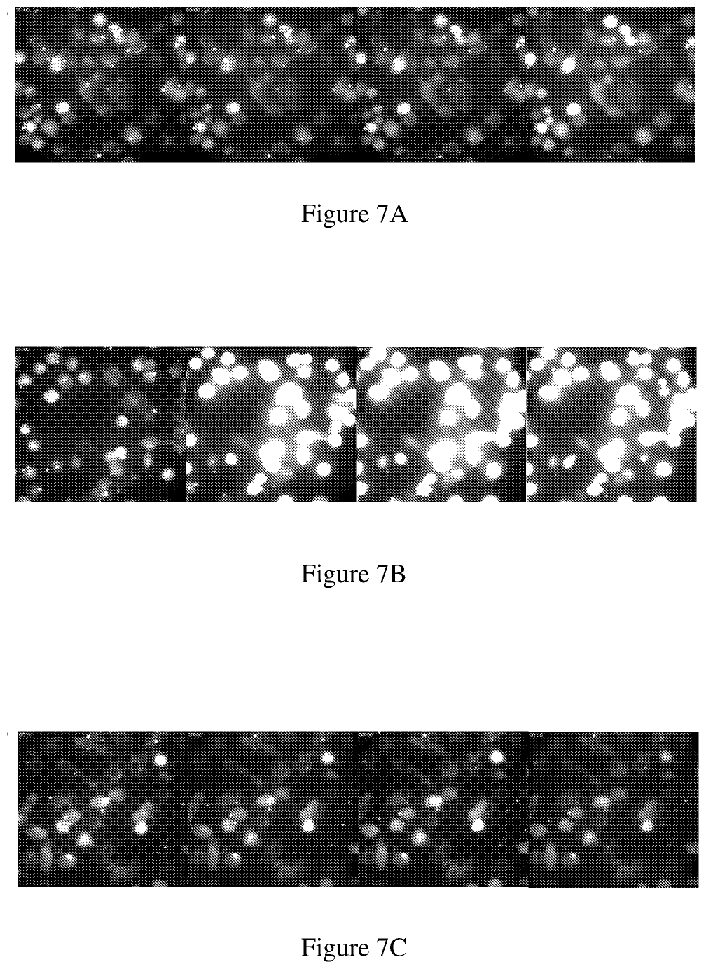

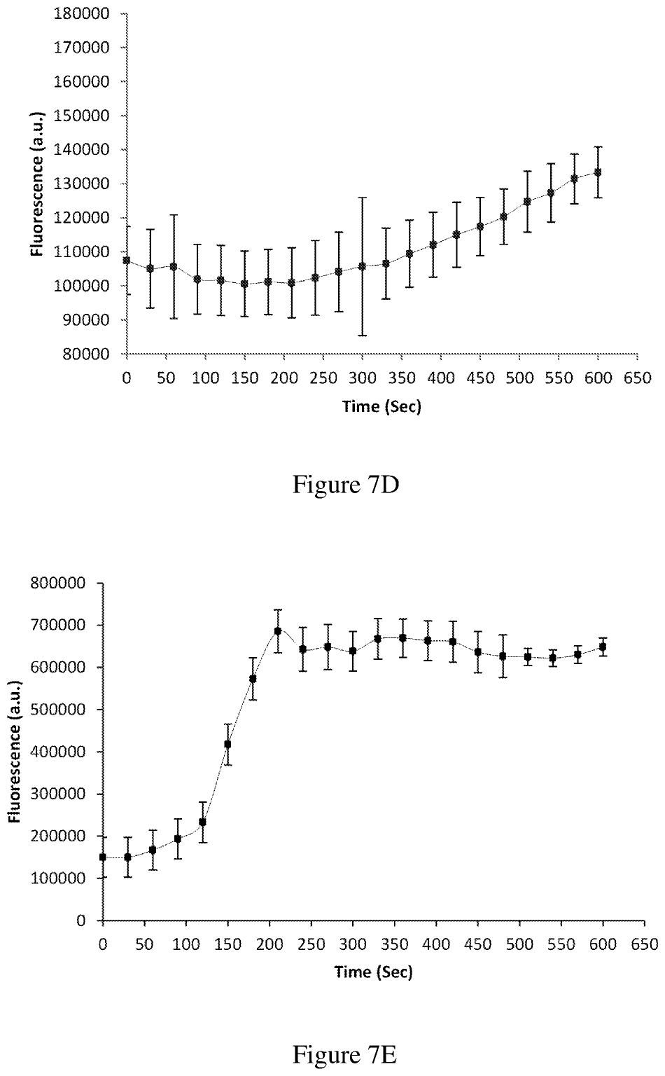

FIGS. 7A-C show live time laps imaging of DU-145 cell line (series ranging from time points t=0, 3, 6 and 10 minutes) incubated with the calcium indicator Fluo-8 AM 4 μM for 30 min prior to washings and the addition of: 2 μM of Corrole 5-Ga/HSA NP's (FIG. 7A), 20 μM of Corrole 5-Ga/HSA NP's (FIG. 7B) and HSA control (FIG. 7C).

Figures D-E and graphs showing the quantification of fluorescence from each cell in the 12 separated fields obtained from series 7A, 2 μM of Corrole 5-Ga/HSA NP's (FIG. 7D) and series 7B, 20 μM of Corrole 5-Ga/HSA NP's (FIG. 7E).

The data points are presented as mean±SEM n=620. Fluorescence was recorded using a ×40 objective and an IN CELL GE analyzer supported with in cell 2000 software. Samples were excited using a FITC filter for Fluo-8 AM detection. Representative images of 12 separate fields, representative results of 3 distinct repeats.

### Example 7: Photodynamic Therapy

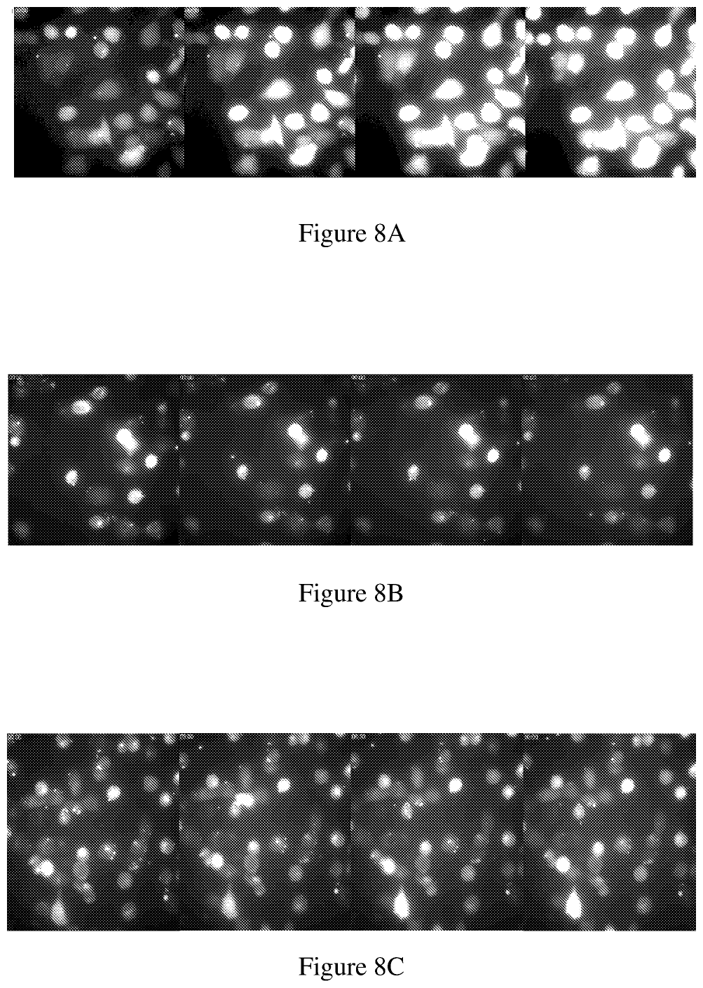

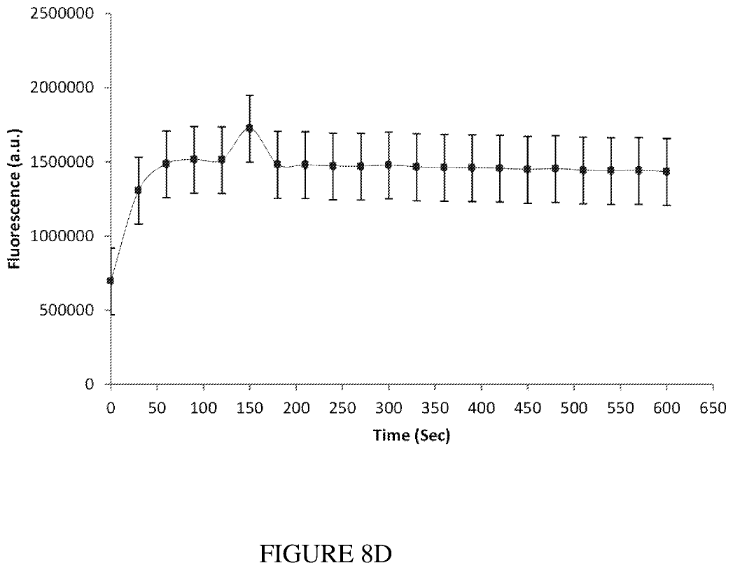

FIGS. 8A-D are live time laps imaging of DU-145 cell line (series ranging from time points t=0, 3, 6 and 10 minutes) incubated with the calcium indicator Fluo-8 AM 4 μM for 30 min prior to washings and the addition of 2 μM of Corrole 5-Ga/HSA NP's followed by excitation of Corrole 5-Ga at each measured time point (FIG. 8A), 2 μM of Corrole 5/HSA NP's (FIG. 8B), 20 μM of Corrole 5/HSA NP's (FIG. 8C), and quantification of calcium indicator Fluo-8 AM fluorescence obtained from each cell in the 12 separated fields of 8A (FIG. 8D). The data points are presented as mean±SEM n=550. Fluorescence was recorded using a ×40 objective and an IN CELL GE analyzer supported with in cell 2000 software. Samples were excited using a FITC filter for Fuo-8 AM detection and a CFP filter for Corrole 5-Ga detection and excitation. Representative images of 12 separate fields, representative results of 3 distinct repeats. Both Corrole 5 and Corrole 5-Ga have low activity at the given doses, while excitation increases activity at low doses is lower than the detected IC50 value of Corrole 5-Ga—5 μM.

### Example 8: Phosphorous MRI, 67-Gallium Imaging and Targeting of (4)Ga

### Example 8A: Specificity of (4)Ga Intake into Cancer Cells

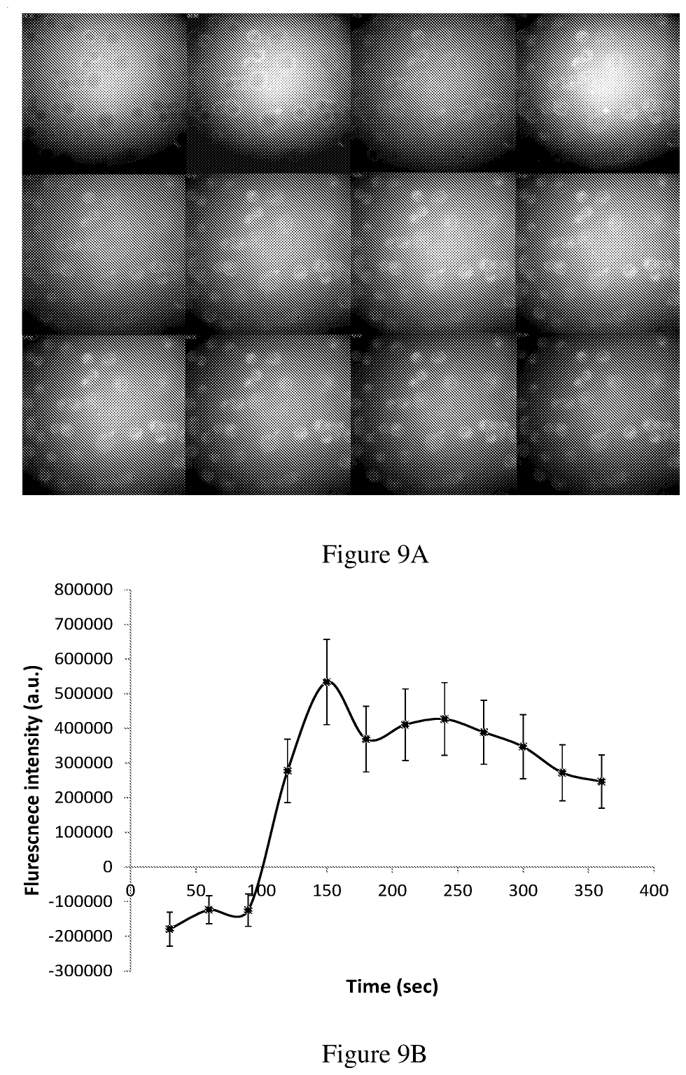

FIGS. 9A-B are live time laps imaging of Corrole 5-Ga/HSA NP's uptake in to DU-145 (prostate cancer) cell line: series of images taken at time intervals of 30 sec raging from t=0 upper left corner to t=6:30 min lower right corner, time point is indicated in white on upper left corner of each image (FIG. 9A); quantification of fluorescence from the center of each cell in the 12 separated fields obtained, data points are presented as mean±SEM n=560 (FIG. 9B). Fluorescence was recorded using a ×40 objective and an IN CELL GE analyzer supported with in cell 2000 software. Samples were excited using a CFP filter for Corrole 5-Ga detection. Representative images of 12 separate fields, representative results of 3 distinct repeats.

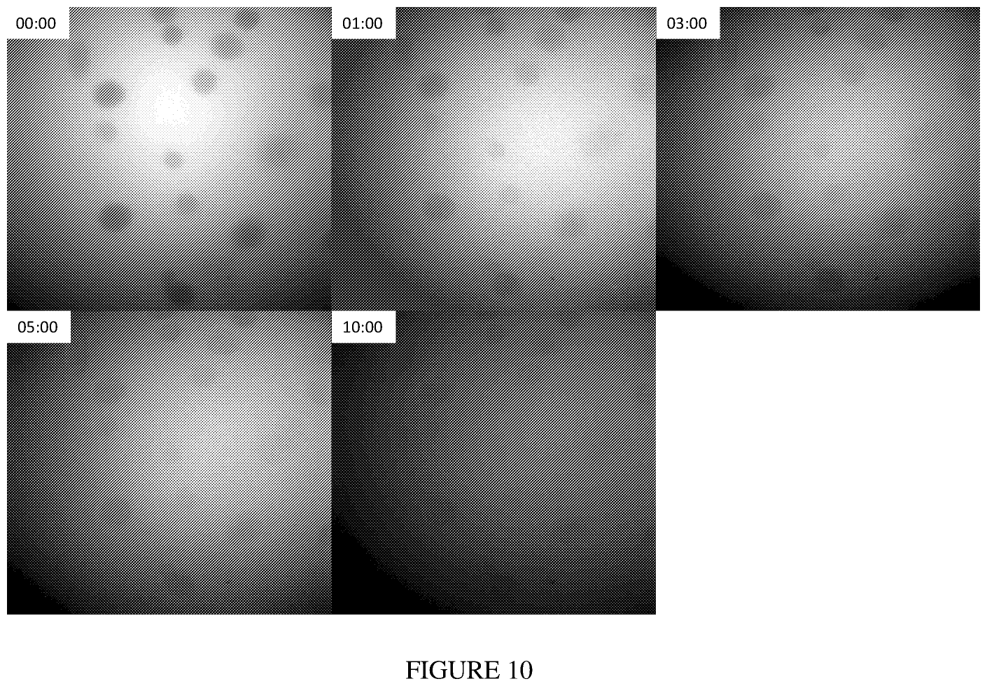

FIG. 10 shows live time laps imaging of Corrole 5/HSA BNC's uptake in to DU-145 cell line: series of images (originally taken at time intervals of 30 sec) raging from t=0 upper left corner to t=10 min lower right corner, time point is indicated in white on upper left corner of each image; representative images of chosen time points. Fluorescence was recorded using a ×40 objective and an IN CELL GE analyzer supported with in cell 2000 software. Samples were excited using a CFP filter for Corrole 5 detection. Representative images of 12 separate fields, representative results of 3 distinct repeats.

Since no uptake of Corrole 5 could be detected, selectivity of gallium-based derivative is proved.

### Example 8B: Examples for MRI/Gallium Imaging Active Corrole 5-M Derivatives

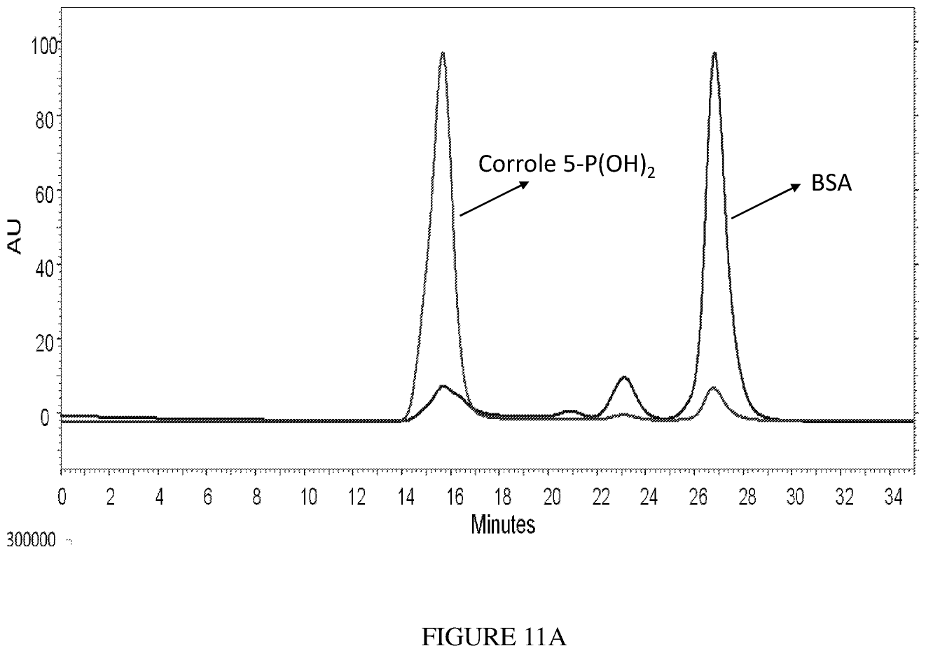

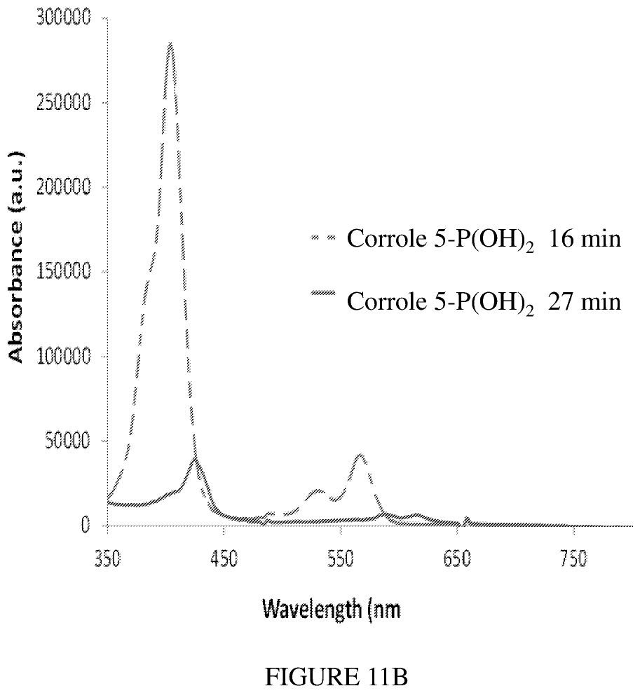

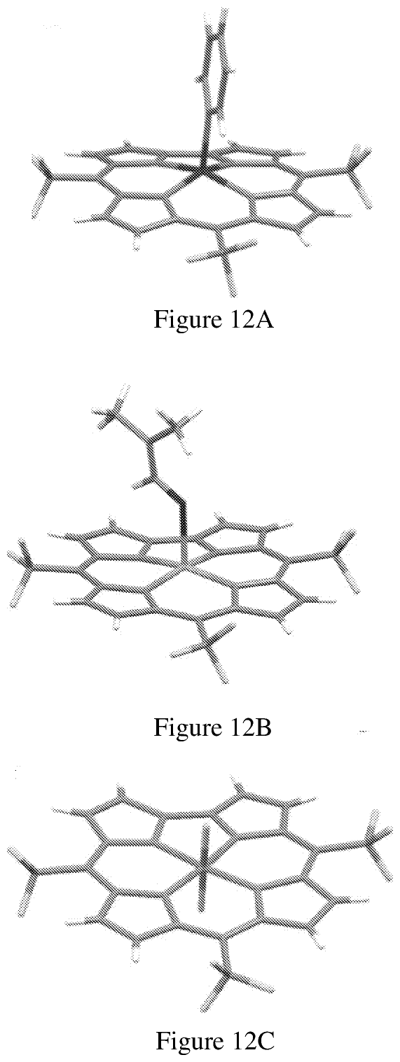

FIGS. 11A-B are HPLC chromatograms with reading set at 280 nm for detection of BSA and at 416 nm for detection of the Corrole 5-P(OH)2 (FIG. 11A) and UV-vis spectra recorded for the HPLC fractions obtained for the Corrole 5-P/HSA conjugate (FIG. 11B).

The corrole 5-P(OH)2 forms nanoparticles as in concordance with other mentioned lipophilic corroles. Corrole 5-P with either OH or 18F can be used for 31P MRI imaging and positron emission tomography imaging respectively.

Relative to Corrole 5-Ga, Corrole 5-P forms much more particles. That is, more corrole is distributed to the early eluting fraction than to the later 27 min fraction.

FIGS. 12A-C are crystal structures of Corrole 5-Ga (pyridine) (FIG. 12A), Corrole 5-Mn (DMF) (FIG. 12B), and Corrole 5-P (F2) (FIG. 12C).

### Example 9: Therapeutic Effect In Vivo

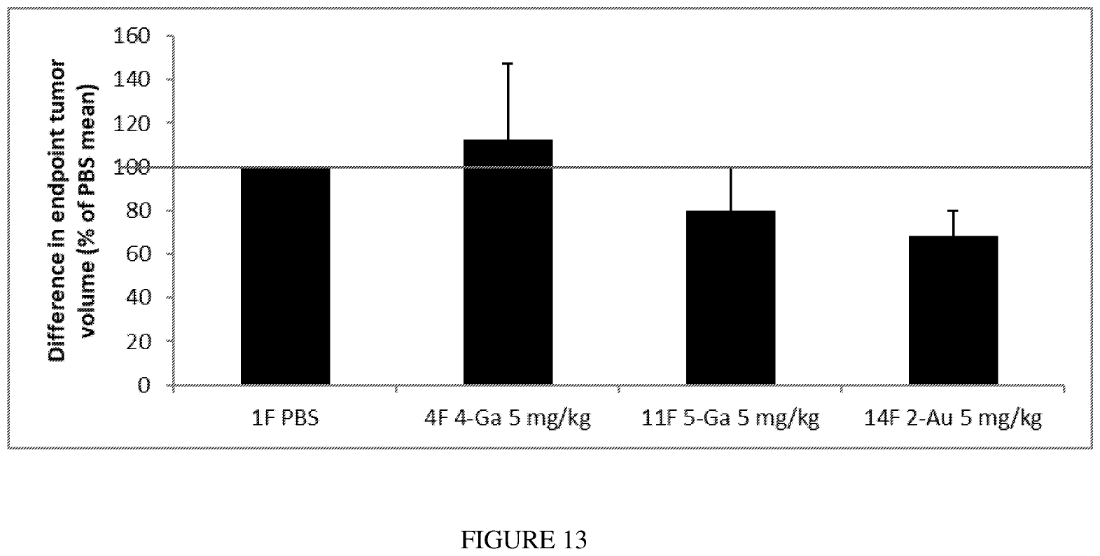

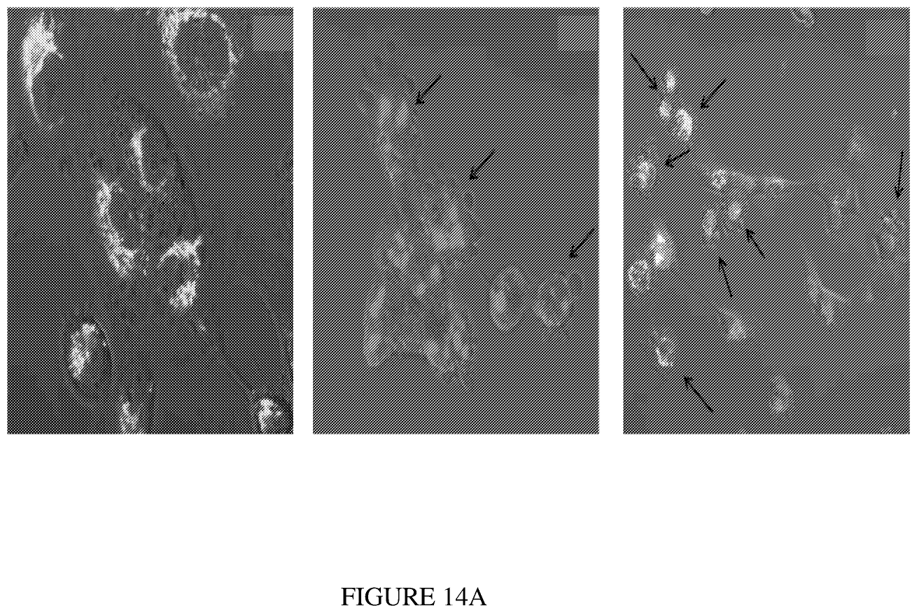

FIG. 13 shows the In Vivo efficacy against a triple negative cell line MDA-MB-231 implanted in nude mice. Triple-negative breast cancer is an aggressive form of breast cancer with limited treatment options. The inventors disclose a therapeutic example of corrole 2-Au and 5-Ga with more impressive activity of the former. Corrole 2-Au disclose close to a 50% reduction in tumor volume relative to 20 to 30 percent in 5-Ga treated specimens. As expected corrole 4-Ga show no activity against this cell line, with a slight increase in tumor volume relative to control.

### Example 10: Apoptosis Tests

In exemplary procedures, upon administration of Corrole 5-Ga conjugated to albumin, induction of persistent calcium influx was apparent which induce eventual apoptosis.

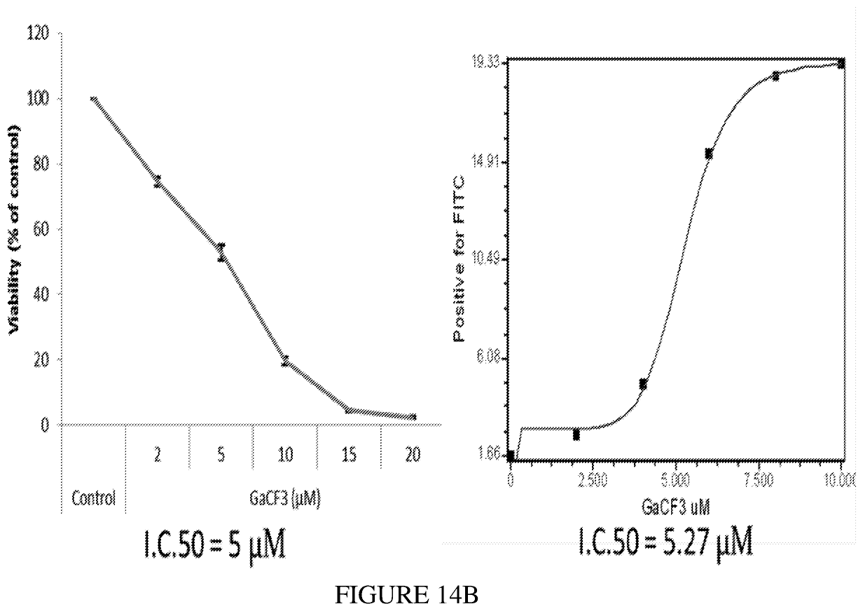

FIG. 14A presents fluorescence examination showing uptake of 5-Ga albumin base nanoparticles by DU-145 prostate cell line dyed with 2 μM mitotracker green (MTG) imaged after 2 h incubation (black arrows designated apoptotic cells with obvious apoptotic blabbing phenotype): control (left panel, “a”); 5-Ga 20 μM, depolarization of mitochondria is apparent (indicative of apoptosis) due to no fluorescence from MTG, lysosomal fusion with the contained compound is also apparent (also indicative of apoptosis) (middle panel, “b”); 5-Ga 5 μM, abnormal mitochondrial signal is apparent with some co-localization of 5-Ga with MTG (yellow signal) (right panel, “c”); and FIG. 14B presents the corresponding IC50 values of 5-Ga added dissolved in DMSO only assessed by MTT test (left panel, “a”) and annexin-FITC apoptotic assay kit (absolute proof of apoptosis) after 24 h incubation (right panel, “b”).

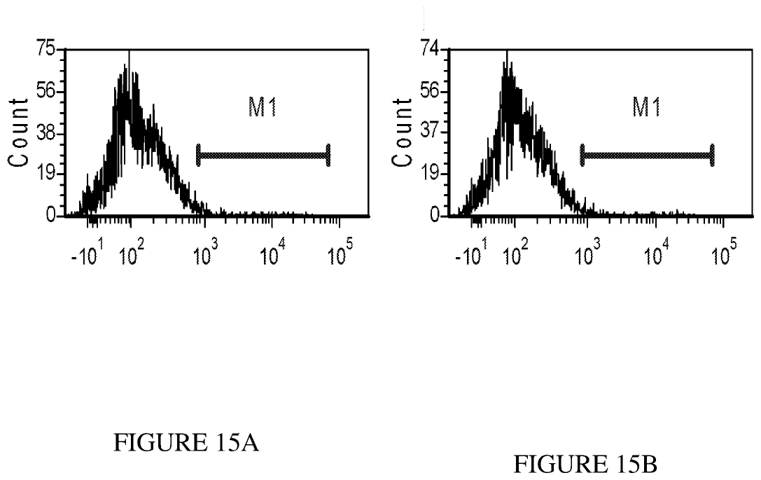

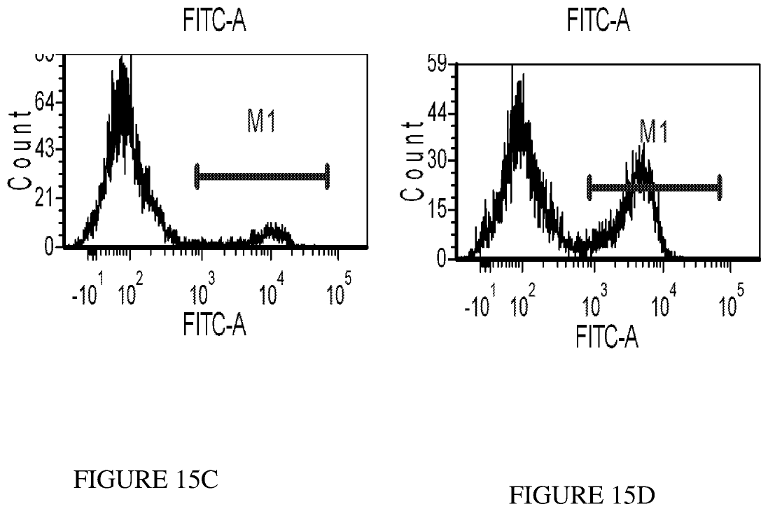

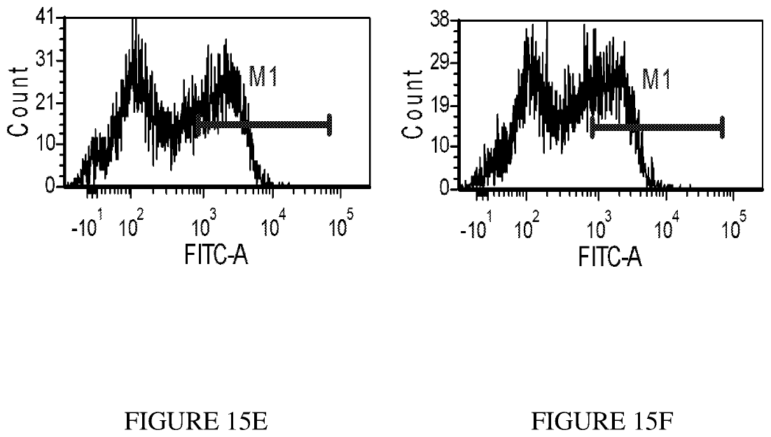

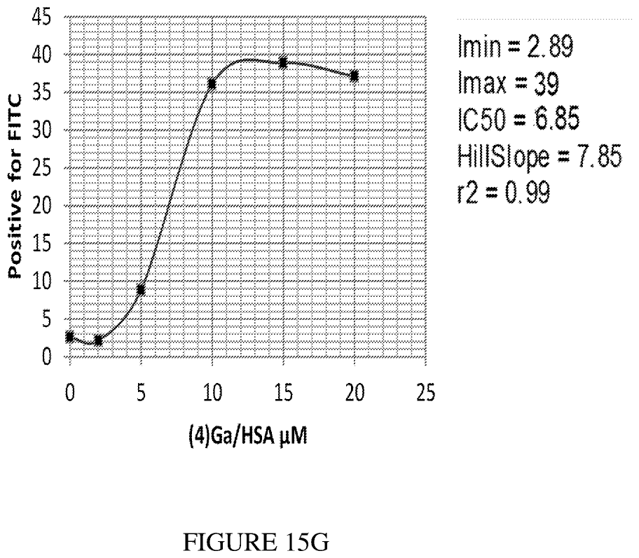

FIGS. 15A-F present FACS analysis of DU-145 prostate cancer cell line treated with an annexin V-FITC kit after 4 h incubation with: HSA control; FIGS. 15B-F present 5-Ga HSA NP's 2, 5, 10, 15, 20 μM respectively. FIG. 15G presents Plotting of gated cells (within Ml) positive to FITC (i.e., apoptosis) against 5-Ga concentration and assessment of IC50 value (IC50 =6.85 μM)

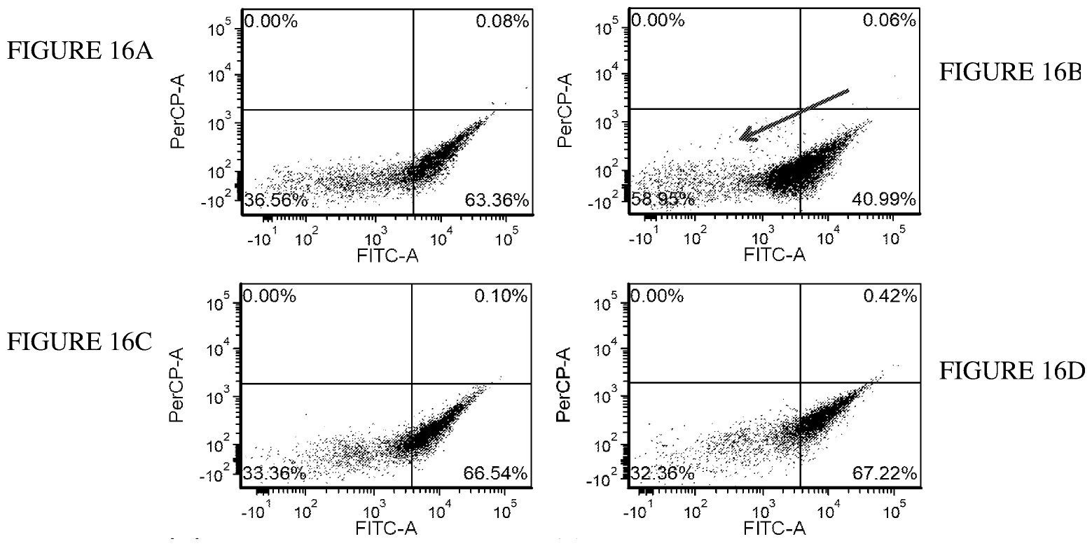

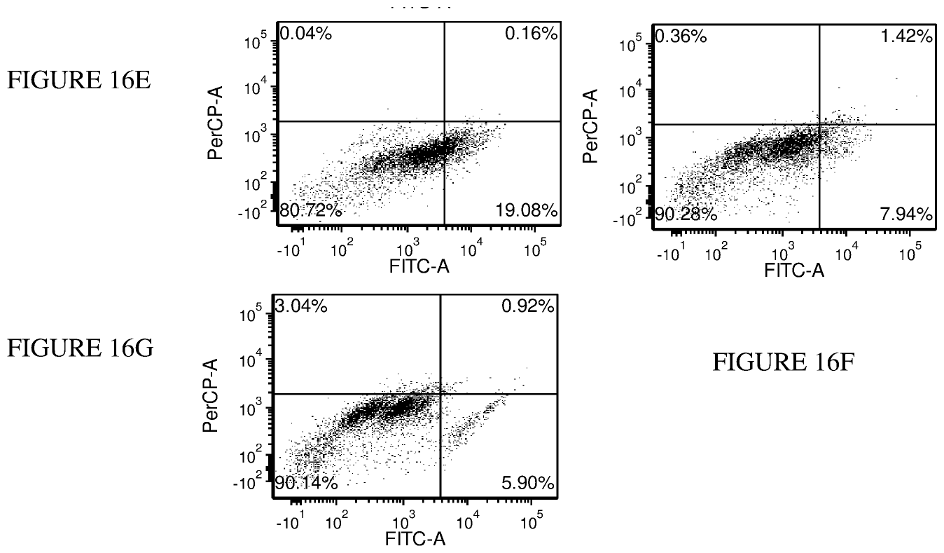

FIGS. 16A-G present FACS analysis of DU-145 prostate cancer cell line treated with a mitochondrial depolarization reporter (another indication for apoptosis) kit after 4 h incubation with: HSA control (FIG. 16A); CCCP (positive control which induces mitochondrial depolarization) (FIG. 16B); and 5-Ga NP's 2, 5, 10, 15, 20 μM, respectively (FIG. 16C-G). Note arrow pointing towards the right quarter that indicates percentage of depolarization.

Table 3 below presents IC50 values of the corrole 1 series, added as is in DMSO solutions, recorded for different cancer cell lines using the MTT test. It is noteworthy that no therapeutic activity of the corrole 1 chelates (very high IC50 values) could be detected when it is added using DMSO (not the case with 5-Ga which is the only corrole which exhibited the same activity even without the protein binding formulation).

TABLE 3

| | Corrole 1 series (μM) IC₅₀ | | | | | |
|---|---|---|---|---|---|---|
| Cell line | 1-Ga | 1-Au | 1-Sb | 1-Fe | 1-Mn | 1-Al |
| DU145 | 156.6 | >400 | >400 | 297.9 | >400 | >400 |
| SK-MEL-28 | 171.5 | >400 | >400 | 116.4 | >400 | >400 |
| MDA-MB-231 | 177.1 | >400 | 296.5 | 115.7 | 129.1 | >400 |
| OVCAR-3 | 110.5 | >400 | >400 | 352.6 | >400 | >400 |

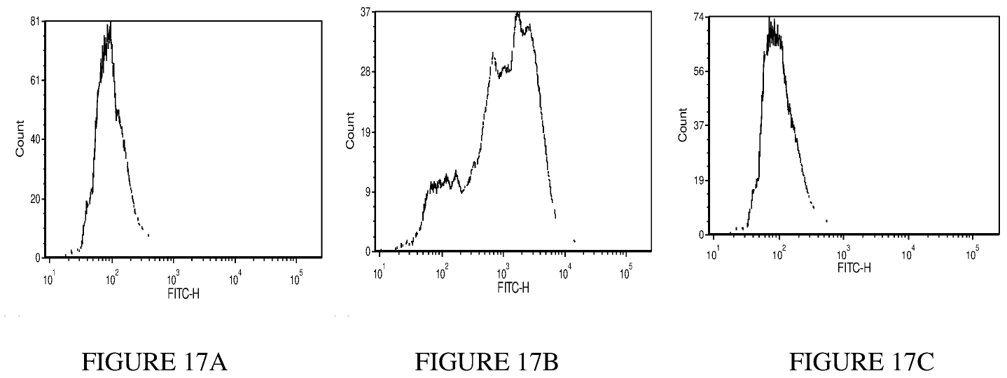

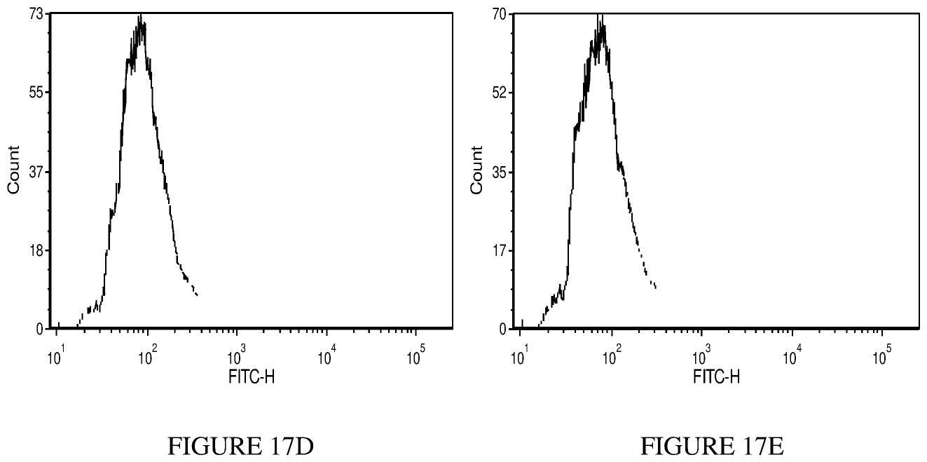

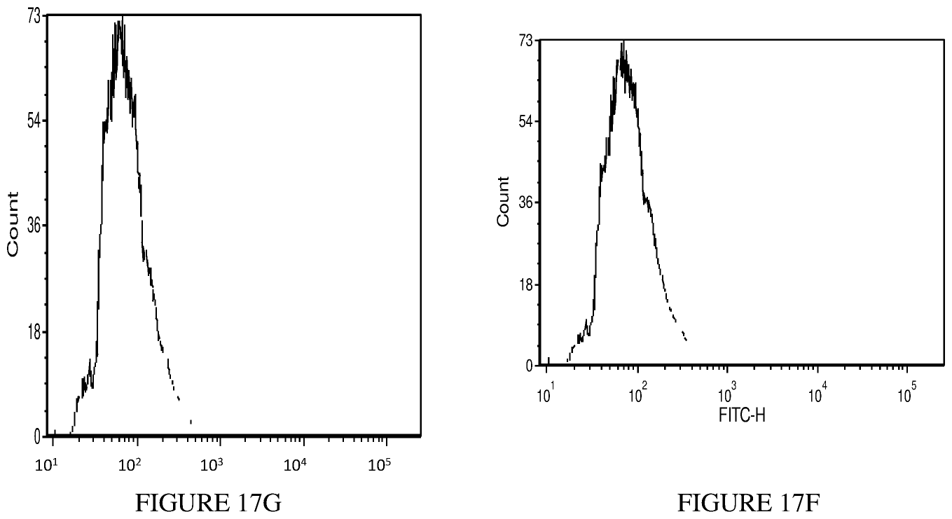

FIGS. 17A-G present FACS analysis of DU-145 prostate cancer cell line treated with an annexin V-FITC kit after 24 h incubation with: (FIG. 17A) BSA control; (FIG. 17B) 0-lapachone (a known inducer of apoptosis, note the strong shift of the distribution curve towards stronger mean fluorescence); and (FIGS. 17C-G) 1-Ga HSA NP's 0.001, 0.01, 0.1, 1, 10 μM, respectively. Analysis of β-lapachone was done after 4 h since it induces apoptosis faster. In contrast with 5-Ga, even when the protein formulation is used there is no induction of apoptosis with 1-Ga (The same is when it is added using DMSO, Table 3).

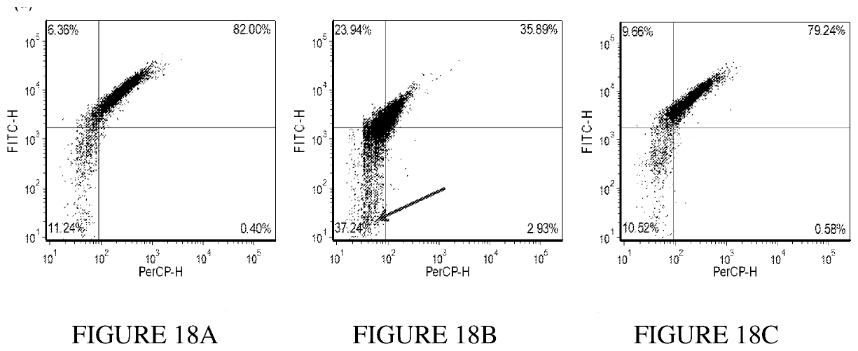

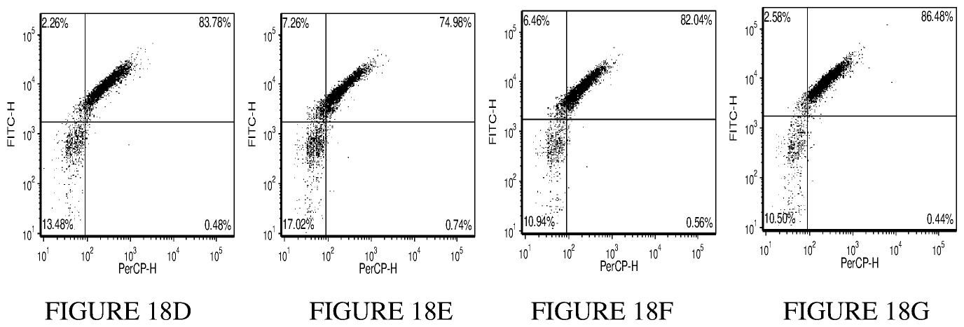

FIGS. 18A-G present FACS analysis of DU-145 prostate cancer cell line treated with a mitochondrial depolarization reporter kit (similar to FIGS. 16A-G) after 24 h incubation with: BSA control (FIG. 18A); CCCP (positive control) (FIG. 18B); and 1-Ga HSA NP's 0.001, 0.01, 0.1, 1, 10 μM, respectively (FIGS. 18C-G). Note red arrow pointing towards the right quarter that indicates percentage of depolarization. In contrast with 5-Ga, no apparent mitochondrial depolarization could be detected even under the use of the aforementioned formulation.

Taken together, the formation of HSA particles with 5-Ga provides an advantageous effect in terms of systemic toxicity, pharmacokinetics parameters and bioavailability when it is administered in vivo. Nonetheless the corrole molecule showed activity on its own (bound or unbound to proteins).

In the description and claims of the present application, each of the verbs, “comprise” “include” and “have,” and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. A composition comprising:
a corrole according to formula [I]; 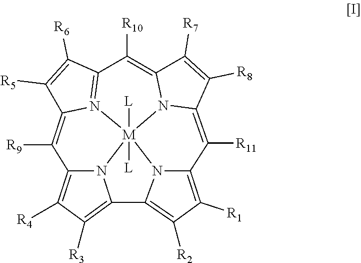
wherein R₁ through R₈ are each independently H, halogen, CHO, C₁-C₄ alkyl or halogenated alkyl;
R₉ through R₁₁ are each independently H, straight or branched C₁-C₁₂ alkyl, C₆-C₁₂ aralkyl, C₆-C₁₂ aryl, or C₆-C₁₂ heteroaryl, having an oxygen or nitrogen atom as a heteroatom, wherein each of straight or branched C₁-C₁₂ alkyl, C₆-C₁₂ aralkyl, C₆-C₁₂ aryl, or C₆-C₁₂ heteroaryl may be unsubstituted or substituted by a halogen or by an amino group;
L, may be present or absent, if present is halogen, an oxo group, a hydroxyl group, acetonitrile, dimethylsulfoxide, dimethylformamide, a pyridine group or a water molecule;
and
M is H or an atom from group 3-15 or an f-block element;
and
a protein non-covalently conjugated to the corrole, the composition having the form of nanoparticles, wherein at least 50% of said particles have a mean diameter size in the range of 30 nm to 200 nm.

2. The composition according to claim 1 wherein said corrole is hydrophobic.

3. The composition according to claim 1, wherein said corrole has a solubility in water of less than 0.1 mg/L.

4. The composition according to claim 1 wherein R₁-R₈ are each independently H, halogen, C₁-C₄ alkyl or halogenated alkyl.

5. The composition according to claim 1 wherein R₁-R₈ are each independently CHO, hydrogen or halogen.

6. The composition according to claim 1 wherein R₉-R₁₁ are each independently C₆ aryl, substituted by one or more halogen groups and/or amino groups.

7-8. (canceled)

9. The composition according to claim 1 wherein M is an f-block or a d-block element.

10. The composition according to claim 1 wherein M is selected from the group consisting of: Sn, Al, Ga, Mn, Gd, Fe, Au, P and Al.

11. The composition according to claim 1 wherein the molar ratio between the protein and the corrole is between 100:1 and 0.1:1.

12. The composition according to claim 1 wherein the protein is a plasma protein.

13. The composition according to claim 12 wherein the plasma protein is selected from the group consisting of: albumin, lipoprotein, glycoprotein, and α, β, and γ globulin, and transferrin.

14. (canceled)

15. The composition according to claim 1 wherein R₉-R₁₁ are each C₆F₅ and R₁-R₈ are each H.

16.-17. (canceled)

18. The composition according to claim 1 wherein R₉-R₁₁ are each CF₃ and R₁-R₈ are each H, and M is Ga, Mn, or Fe.

19-24. (canceled)

25. A method for the manufacture of a composition according to claim 1 comprising:
combining an aqueous solution of a plasma protein with a solution of a corrole according to formula [I].

26. (canceled)

27. A method for treating a disease in a subject in need thereof, comprising:
administering a therapeutically effective amount of the composition of claim 1 to said subject; thereby treating a disease.

28. The method of claim 27, wherein said disease is cancer.

29. The method of claim 27, wherein said disease is associated with a reactive oxygen species.

30. The method of claim 27 for photodynamic therapy (PDT), sonodynamic therapy (SDT) or a combination thereof, further comprising irradiating said subject with energy source at a wavelength capable of exciting the composition.

31-35. (canceled)

36. The method of claim 27, wherein said disease is a disease which can be modulated by induction of membranal destabilization or calcium influx to induce apoptosis.

37. The method of claim 27 for treatment of cancer in further comprising sonicating said subject with energy source at sufficient frequency and conditions capable of exciting the composition.
